(19) United States
(12) Patent Application Publication
(10) Pub. No.: US 2025/0099595 A1
(43) Pub. Date: Mar. 27, 2025

(54) HYDROGEL-BASED DELIVERY SYSTEM FOR INDUCTION OF INTRATUMORAL TERTIARY LYMPHOID STRUCTURES AND AUGMENTATION OF IMMUNE CHECKPOINT BLOCKADE AND METHODS THEREOF

(71) Applicants: THE UNIVERSITY OF HONG KONG, HONG KONG (CN); UNIVERSITY OF NOTRE DAME DU LAC, SOUTH BEND, IN (US)

(72) Inventors: Zhou YE, HONG KONG (CN); Jiandong HUANG, HONG KONG (CN); Chaiyaporn KUWENTRAI, HONG KONG (CN); Weilong TANG, HONG KONG (CN); Matthew WEBBER, GRANGER, IN (US)

(73) Assignees: THE UNIVERSITY OF HONG KONG, HONG KONG (CN); UNIVERSITY OF NOTRE DAME DU LAC, SOUTH BEND, IN (US)

(21) Appl. No.: 18/891,525

(22) Filed: Sep. 20, 2024

Related U.S. Application Data
(60) Provisional application No. 63/584,248, filed on Sep. 21, 2023.

(51) Int. Cl. A61K 47/54; A61K 9/00; A61K 38/17; A61K 38/19; A61K 39/00; A61K 47/69; A61P 35/00; C07K 16/28

(52) U.S. Cl. CPC A61K 47/549; A61K 9/0019; A61K 38/177; A61K 38/19; A61K 47/545; A61K 47/6903; A61P 35/00; C07K 16/2818; A61K 2039/505

(57) ABSTRACT

Provided is a system for drug delivery utilizing hydrogel to deliver biologically active agents to stimulate mature intratumoral tertiary lymphoid structure formation and augment immune check point blockade. Provided herein is an injectable hydrogel that can stimulate the formation of immune structures within tumors, improving cancer prognosis and treatment response. Provided is a method of treating cancer. Also provided is a method of making the drug delivery system.

### RELATED APPLICATION

The present application claims priority to U.S. provisional application Ser. No. 63/584,248 filed Sep. 21, 2023, which is incorporated by reference in its entirety.

### 1. FIELD

Disclosed is a hydrogel delivery system in tumors for immunotherapy to improve cancer prognosis and treatment response.

### 2. BACKGROUND

The World Health Organization (WHO) has estimated that there are approximately 2 to 3 million annual cases of skin cancer worldwide. Melanoma, the most dangerous type of skin cancer, accounts for 132,000 of these cases and is responsible for the majority of skin cancer fatalities¹. Therapeutic interventions for advanced-stage melanoma are challenging due to the high likelihood of disease recurrence². Over the past decade, various immunotherapies, including cytokines/chemokines, immune-checkpoint blockage (ICB), and their combinations, have been explored to address this challenge³. However, these immunotherapies have limitations, such as off-target toxicity and low patient response rates⁴. The limited drug response is associated with the suppressive tumor microenvironment, which results in scarce tumor-infiltrating T cells⁵⁻⁶. Therefore, therapeutic advances designed to enhance protective immunity against tumors are of clinical significance.

Tertiary lymphoid structures (TLSs) are ectopic organizations of lymphoid immune cells that stimulate antigen-driven responses during states of chronic inflammation⁷⁻⁸. In disease-induced inflammation, TLSs resemble the partial formation of secondary lymphoid organs, such as the spleen, within non-lymphoid tissue⁹. TLSs are immune niches composed of various immune cell types, including B cells, T cells and antigen-presenting cells, that drive protective responses against tumor progression¹¹. The beneficial prognosis associated with TLSs can be attributed to four main mechanisms: expedited immune responses, effective interactions between antigens and lymphocytes, direct contact established between lymphocytes and tumor cells, and lymphocyte homeostasis facilitated by secreted survival factors¹²⁻¹⁴.

Previous studies have investigated the cellular and molecular mechanisms underlying TLSs formation, with the B cell pathways involving the CXC chemokine ligand 13/CXC chemokine receptor type 5 axis and the CC chemokine ligand (CCL)19/CCL21/CC-chemokine receptor 7 axis playing a vital role in TLSs generation¹⁶. Furthermore, the interaction between LTα and LTi cells initiates cell recruitment via cytokines and chemokines signaling to produce TLSs¹⁷. Critical factors involved in the process include CXCL13, CCL21, IL-4, IL-7, IL-2 and LIGHT¹⁴. Chemokine CXCL13 has been implicated as a TLSs-inducing factor in tumors, recruiting B cells into B cell-rich structures that facilitate TLSs formation¹⁸. This chemokine also serves as a potential biomarker for TLSs formation within tumor microenvironments¹⁹. Alternatively, the cytokine LIGHT is another bona fide TLSs inducer²⁰. LIGHT has been successfully applied in pilot studies to induce TLSs and promote the generation of effector and memory T cells when combined with immunotherapies in mouse models²¹⁻²². However, there is currently a lack of clarification regarding which factors are critical for upregulating TLSs density and maturity within tumor microenvironments²³⁻²⁴.

Previously, TLSs was induced through direct intratumoral injection of cytokines, chemokines, antibodies, or antigen-presenting cells²⁵. Due to the high cost of injecting antigen-presenting cells, more research focuses on directly injecting cytokines/chemokines, including the aforementioned CXCL13 and LIGHT²⁶. However, these approaches have limitations, such as high dose-dependency of cytokines/chemokines for inducing TLSs, generation of immature and imperfect TLS structures, and reduced interaction and activity between B cells and T cells²⁷. Consequently, there is a growing need to develop new methods for the sustained delivery of cytokines/chemokines and controlled infiltration and organization of immune cells²⁸.

Compared to other biomaterials, hydrogels are non-invasive application, minimally invasive nature, and low technical sensitivity²⁹. Hydrogels are typically constructed from a network of highly hydrophilic polymers through chemical or physical cross-linking³⁰. In contrast to stable, irreversible, and permanent chemical cross-linking, physical cross-linking is transient, reversible, and dynamic. Hydrogels formed through physical cross-linking exhibit injectability and self-healing characteristics due to their non-covalent bonding properties³¹. Additionally, drug release from hydrogels can be controlled by adjusting the hydrogel's mesh size, swelling ratio, and degradation rate³².

The problem that this patent application seeks to solve is the varied outcomes of current cancer immunotherapies, such as cancer vaccines or immune checkpoint blockade (“ICB”), in patients. This inconsistency in treatment efficacy can be attributed to some tumors being “immunologically cold tumors” with low immune responses. Therefore, there is a need to modify the immune tumor microenvironment to stimulate the immune system so as to enhance the effectiveness of immunotherapies.

### 3. SUMMARY

The present disclosure implemented a pioneering drug delivery approach utilizing a physically cross-linked hydrogel to deliver CXCL13 and LIGHT proteins, aiming to stimulate mature TLSs formation and augment ICB treatment in melanoma. Provided herein is a hydrogel-based delivery system composed of cytokines and chemokines for induction of intratumoral tertiary lymphoid structures (“TLSs”) and augmentation of immune checkpoint blockade. In the past, cytokines/chemokines were indirectly injected to induce TLSs, but the generated TLSs are immature and not well-structured. The present method uses hydrogels for localized controlled release of cytokines/chemokines to induce mature intratumoral TLSs. The present method uses hydrogels to deliver cytokine/chemokines to induce mature TLS structures and improve ICB efficacy. This is the first time where hydrogel is used to induce TLSs formation.

Provided herein is an injectable hydrogel that can stimulate the formation of immune structures within tumors, improving cancer prognosis and treatment response. In this study, a single injection of the hydrogel, containing specific immune molecules, suppressed tumor growth and increased the density of these immune structures in mouse models. The hydrogel also improved the effectiveness of a specific cancer therapy named immune checkpoint blockade, leading to better survival rates and tumor suppression. Provided herein is a method of using hydrogels as drug carriers to boost the immune system's ability to fight cancer.

In one embodiment, the disclosure leverages the association of TLSs with better prognosis in many cancers and their ability to improve clinical outcomes of immunotherapies. The disclosure utilizes hydrogels to deliver the cytokine LIGHT and the chemokine CXCL13, inducing intratumoral TLSs to enhance the efficacy of immunotherapies.

Provided herein is a compound comprising hyaluronic acid and 4-(4-chlorophenyl)pyridine (HA-CPP) or a salt thereof.

In one embodiment, the compound comprises at least one moiety of formula (1):

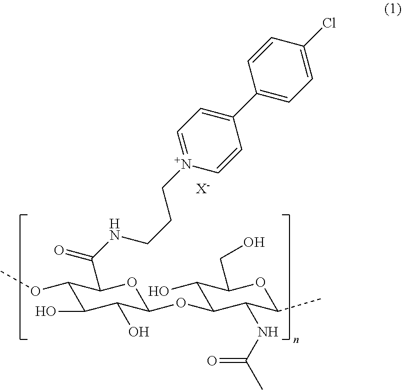

wherein n is an integer and X is a counter-ion;
or a salt thereof.

Provided herein is a compound comprising HA-CPP⊂CB[8] combined with one or more biologically active agent.

In certain embodiments, the HA-CPP⊂CB[8] is combined with the biologically active agent through non-covalent binding to form a complex.

In certain embodiments, the biologically active agent is chemokine, cytokine, immune-checkpoint inhibitor, cancer vaccine, chimeric antigen receptor or a combination thereof.

In certain embodiments, the biologically active agent is CXCL13, CCL21, IL-4, IL-7, IL-2, LIGHT, CCL19, CXCL12, CXCL13, lymphotoxin, TNF-α or a combination thereof.

In certain embodiments, the chemokine is CXCL13 and the cytokine is LIGHT.

Provided herein is a composition comprising the disclosed compound and a pharmaceutically acceptable carrier.

In one embodiment, the pharmaceutically acceptable carrier is water.

In certain embodiments, the composition is an injectable composition and comprises water at a weight ratio of about 1-5%.

In certain embodiments, the composition is administered by injection to form tertiary lymphoid structures and augmentation of immune checkpoint blockade.

Provided is a method of treating cancer comprising administering the disclosed composition to a subject in need thereof.

In certain embodiments, the method suppresses tumor growth, prolongs survival, increases TLS density, promotes TLS maturation, up-regulates OVA antigen-spreading in secondary lymphoid organs or a combination thereof.

In certain embodiments, the method further comprises administration of anti-PD1 drug, anti-CTLA-4 drug, anti-PD1, anti-PDL1, anti-CTLA-4 or a combination thereof.

In one embodiment, the administration of the composition is direct intratumoral injection.

In certain embodiments, the cancer is melanoma, colorectal cancer, lung cancer, pancreatic cancer, oral squamous cell carcinoma, and invasive breast cancer.

In certain embodiments, the subject is human, feline, canine, porcine, rabbit or rodent.

Provided herein is a method of making HA-CPP comprising: (a) reacting 4-(4-chlorophenyl)pyridine with a compound of the formula X—(CH₂)₃—NH₂ wherein X is a leaving group; and (b) reacting the product of step (a) with hyaluronic acid (HA) having molecular weight of about 6 kDa to 1000 kDa.

Provided herein is a method of making HA-CPP comprising: (i) reacting 4-(4-chlorophenyl)pyridine with 3-halopropylamine to form a pyridinium salt CPP—NH₂ and (ii) performing an amidation reaction with at least one glucuronic acid group of hyaluronic acid (HA), wherein the HA has a molecular weight of about 6 kDa to 1000 kDa.

In certain embodiments, the method further comprising combining HA-CPP and cucurbit[8]uril (CB[8]) to form HA-CPP⊂CB[8].

In certain embodiments, the stoichiometric ratio between CPP and CB [8] is 2:1 or greater than 2:1.

### 4. BRIEF DESCRIPTION OF THE FIGURES

FIG. 1: Schematic diagram of mature TLS formation.

FIGS. 2A-2B: Scheme 1 (FIG. 2A) Synthesis route of HA-CPP. (FIG. 2B) Schematic of the formation of HA-CPP⊂CB[8] supramolecular networks.

FIGS. 3A-3C: Chemical structures and 1H NMR spectra of (FIG. 3A) CPP guest, (FIG. 3B) HA-CPP, and (FIG. 3C) complexation of HA-CPP⊂CB[8].

FIGS. 4A-4F: Materials characterizations of HA-CPP⊂CB[8]hydrogel. (FIG. 4A) “HKU” pattern formed by HA-CPP⊂CB[8]hydrogel injected through a syringe needle. (FIG. 4B) Injection of HA-CPP⊂CB[8]hydrogel into deionized water. (FIG. 4C) Shear viscosity of HA-CPP⊂CB[8]hydrogel. (FIG. 4D) Oscillatory rheology frequency sweeps of HA-CPP⊂CB[8]hydrogel. (FIG. 4E) Photographs of two differently stained HA-CPP⊂CB[8]hydrogels self-healed into an integrated piece. (FIG. 4F) G′ and G″ of HA-CPP⊂CB[8]hydrogel in continuous step strain measurements.

FIGS. 5A-5E: In vivo delivery of CXCL13 and LIGHT by HA-CPP⊂CB[8]hydrogel into B16 tumor-bearing mice. (FIG. 5A) Treatment regimen of drug-encapsulated HA-CPP⊂CB[8]hydrogel in B16 mice model. Mice were inoculated with B16-OVA melanoma cell line by intraperitoneal injection at day 0. Intraperitoneal injections of drug-encapsulated hydrogel (Hydrogel+LIGHT+CXCL13), drugs alone (LIGHT+CXCL13), vehicle hydrogel control or PBS control were administered at day 7 (n=5 per group). At the endpoint (day 23), mice were sacrificed for further analysis. For survival experiments, mice were closely monitored until the humane euthanization endpoint (day 40). (FIG. 5B) Primary injection-site tumor sizes of the different experimental groups at the endpoint (day 23). (FIG. 5C) Quantification of primary injection-site tumor sizes of the different experimental groups at the endpoint (day 23). (FIG. 5D) Quantification of total tumor weight of the different experimental groups at the endpoint (day 23). An unpaired t-test was used with multiple t-test adjustments. Data were expressed as mean±SEM. (FIG. 5E) Survival analysis of experimental groups (n=5). The Mantel-Cox test was used. *p<0.05, ns refers to “not significant.”

FIGS. 6A-6E: Drug-encapsulated HA-CPP⊂CB[8]hydrogel induces the formation and maturation of TLSs, and promotes interferon-gamma T cell responses. Immunofluorescence staining and grading of TLSs in the drug-encapsulated HA-CPP⊂CB[8]hydrogel-treated B16 tumors (A-C). (FIG. 6A) Formation of TLSs in the B16 tumor of different experimental groups: Hydrogel+LIGHT+CXCL13 (scale bar-200 μm), LIGHT+CXCL13 (scale bar-200 μm), Control Hydrogel only (scale bar-100 μm). (FIG. 6B) Quantification of TLSs number per mm2 in each experimental cohort. (FIG. 6C) TLSs grading (mature, immature, no TLS) after immunostaining of treated B16 tumors. T cells (CD3 marker), B cells (B220 marker) and antigen-presenting cells (CD11c) were displayed in the tumor microenvironment of the different experimental groups from FIGS. 5B-5E. Kolmogorov Smirnoff test was used to assess the statistical difference. (FIG. 6D) Quantification of TLSs grading percentage in each experimental cohort (n=8 per group). Chi-square contingency analysis was performed. Antigen-activated T cell responses after drug-encapsulated HA-CPP⊂CB[8]hydrogel treatment (FIG. 6D, FIG. 6E). (FIG. 6D) Interferon-gamma ELISPOT results. At day 23, the ELISPOT assay was performed using the splenocytes from the experimental groups in FIG. 5B-5E. OVA-induced splenocytes were analyzed for the interferon-gamma release profiles. (FIG. 6E) Quantification of Interferon-gamma ELISPOT from the different experimental groups (n=4 per group). An unpaired t-test with Welch correction was used for statistical analysis. Data were expressed as mean±SEM. *p<0.05, **p<0.01, ***p<0.001, ****p<0.0001, ns refers to “not significant.”

FIGS. 7A-7E: In vivo delivery of CXCL13 and LIGHT by HA-CPP⊂CB[8]hydrogel in combination with high-dose αPD1 treatment into B16 tumor-bearing mice. (FIG. 7A) Treatment regimen of drug-encapsulated HA-CPP⊂CB[8]hydrogel in combination with high-dose αPD1 treatment in B16 mice model. Mice were inoculated with B16-OVA melanoma cell line by intraperitoneal injection at day 0. Intraperitoneal injections of drug-encapsulated hydrogel (Hydrogel+LIGHT+CXCL13 were administered on day 7 (n=5). At days 14 and 19, αPD1 treatment (0.6 mg per injection) was administered alone or in combination after drug-encapsulated hydrogel treatment (n=5 per group). Vehicle control mice were injected with blank hydrogel and PBS (n=5). At the endpoint (day 23), mice were sacrificed for further analysis. For survival experiments, mice were closely monitored until the humane euthanization endpoint (day 40). (FIG. 7B) Injection-site primary tumor sizes of the different experimental groups at the endpoint (day 23). (FIG. 7C) Quantification of injection-site tumor sizes of the different experimental groups at the endpoint (day 23). (FIG. 7D) Quantification of total tumor weight of the different experimental groups at the endpoint (day 23). An unpaired t-test was used with multiple t-test adjustments. Data were expressed as mean±SEM. *p<0.05, **p<0.01, ns refers to “not significant.” (FIG. 7E) Survival analysis of experimental groups (n=5). The Mantel-Cox test was used. *p<0.05, ns refers to “not significant.”

FIGS. 8A-8E: Analysis of TLSs and T cell infiltrates in combination-treated B16 tumor-bearing mice. (FIG. 8A) TLSs formation was observed after immunostaining of B16-treated tumors. T cells (CD3 marker), B cells (B220 marker) and antigen-presenting cells (CD11c) were observed in the tumor microenvironment of the different experimental groups from FIGS. 7B-7E. Mature TLSs were observed in the combination group (scale bar=200 μm). Immature TLSs or no TLS were observed in the αPD1 group (scale bar=200 μm) and vehicle control groups (50 μm), respectively. (FIG. 8B) Quantification of TLSs number per mm2 in each experimental cohort. Kolmogorov Smirnoff test was used for statistical analysis. (FIG. 8C) Quantification of TLSs grading percentage in each experimental cohort (n=5 per group), Chi-square contingency analysis was performed. (FIG. 8D-8E) Flow cytometric analysis of (FIG. 8D) CD3+CD8+ T cell populations and (E) CD3+CD4+ T cell populations in the B16 tumors of each experimental cohort. (FIGS. 8F-8G) Flow cytometric analysis of (FIG. 8F) CD3+CD8+ T cell populations and (FIG. 8G) CD3+CD4+ T cell populations from the spleen of each experimental cohort. Experiments were performed in triplicates. An unpaired t test with Welch correction was performed. Data were expressed as mean±SEM. **p<0.01, ***p<0.001, ****p<0.0001, ns refers to “not significant.”

FIGS. 9A-9B Hydrogel-based delivery of LIGHT+CXCL13 in B16-tumor bearing mice. (FIG. 9A) Schematic of hydrogel vaccine administration after intraperitoneal B16 tumor inoculation. Hydrogel delivered 0.6 μg LIGHT and 0.6 μg CXCL13 per animal via intraperitoneal injection. (FIG. 9B) Serum ELISA of vaccinated mice on day 14. ELISA plate was precoated with HA-CPP⊂CB[8]hydrogels to detect specific antibody reaction in mice serum. ns refers to “not significant”.

FIGS. 10A-10C Hydrogel-based delivery of LIGHT in B16-tumor bearing mice. (FIG. 10A) Schematic of hydrogel vaccine administration after intraperitoneal B16 tumor inoculation. Hydrogel delivered 0.6 μg LIGHT per animal via intraperitoneal injection. (FIG. 10B) Primary injection-site tumor was recorded using electronic callipers. (FIG. 10C) Total tumor per mice were weighed and compared between different groups. ns refers to “not significant”.

FIG. 11 Multicolor staining of Hydrogel+LIGHT treated B16 tumors. Multicolor staining of Hydrogel+LIGHT treated B16 tumors (CD3, B220, CD11c, DAPI). Scale bars are 200 μm.

FIG. 12 Infiltration of splenocytes into hydrogel. Cells were seeded in the cell plates and cultured with hydrogel. Cell infiltration into the hydrogel was observed after 1 day and 2 days by light microscopy. The scale bars are 0.5 mm (left) and 1 mm (right). Quantification of spleen cells per area (mm2) on day 2. **p<0.01.

FIGS. 13A-13K. In vivo delivery of CXCL13 and LIGHT mRNA by HA-CPP⊂CB[8]hydrogel into B16.f10 tumor-bearing mice, (FIG. 13A) Treatment regimen of HA-CPP⊂CB[8]hydrogel-loaded with liposome-encapsulated CXCL13 and LIGHT mRNA in B16.f10 mice model. Mice were inoculated with B16.f10 melanoma cell line by subcutaneous injection at day 0. Intratumoral injections of drug-encapsulated hydrogel (Hydrogel-LIGHT+CXCL13 mRNA), drugs alone (LIGHT+CXCL13 mRNA), vehicle hydrogel control or vehicle liposome control were administered at day 5, 8, and 11 (n=5 per group). At the endpoint (day 23), mice were sacrificed for further analysis. For survival experiments, mice were closely monitored until the humane euthanization endpoint (day 40). (FIG. 13B) Individual tumor growth kinetics of the different experimental groups. (FIG. 13C) Primary injection-site tumor sizes of the different experimental groups at the endpoint (day 23). (FIG. 13D) Quantification of primary injection-site tumor sizes of the different experimental groups at the endpoint (day 23). An unpaired t-test was used with multiple t-test adjustments. Data are expressed as mean±SEM. (FIG. 13E) Survival analysis of experimental groups (n=5). The Mantel-Cox test was used. *p<0.05, ns refers to “not significant,” Immunofluorescence staining and grading of TLSs and immune cell densities in the drug-encapsulated HA-CPP⊂CB[8]hydrogel-treated B16.f10 tumors (F—K). (FIG. 13F) Formation of TLSs in the B16.f10 tumor of different experimental groups: Hydrogel-LIGHT+CXCL13 mRNA (scale bar=200 μm), LIGHT+CXCL13 mRNA (scale bar=100 μm), Control Hydrogel only (scale bar=100 μm), T-cells (CD3 marker), B-cells (B220 marker) and antigen-presenting cells (CD11c) were displayed in the tumor microenvironment of the different experimental groups. (FIG. 13G) Quantification of TLSs number per mm² in each experimental cohort. Kolmogorov Smirnoff test was used to assess the statistical difference. (FIG. 13H) Formation of mature germinal center-like (Ki67+) TLS in the Hydrogel-LIGHT+CXCL13 mRNA group (scale bar=100 μm) and immature TLS in the LIGHT+CXCL13 mRNA group (scale bar-100 μm). (FIG. 13I) TLSs grading (mature, immature, no TLS) after immunostaining of treated B16.f10 tumors (n=5 per group). Chi-square contingency analysis was performed. (FIG. 13J) Quantification of B220+ B cells per mm² from the tumors of various experimental groups. (FIG. 13K) Quantification of CD3+ T cells per mm² from the tumors of various experimental groups. An unpaired t-test with Welch correction was used for statistical analysis. Data were expressed as mean±SEM. *p<0.05, **p<0.01, ***p<0.001, ****p<0.0001, ns refers to “not significant”.

FIGS. 14A-14J. In vivo delivery of CXCL13 and LIGHT mRNA by HA-CPP⊂CB[8]hydrogel into CT26 tumor-bearing mice. (FIG. 14A) Treatment regimen of HA-CPP⊂CB[8]hydrogel-loaded with liposome-encapsulated CXCL13 and LIGHT mRNA in CT26 mice model. Mice were inoculated with CT26 colon cancer cell line by subcutaneous injection at day 0. Intratumoral injections of drug-encapsulated hydrogel (Hydrogel-LIGHT+CXCL13 mRNA), drugs alone (LIGHT+CXCL13 mRNA), vehicle hydrogel control or vehicle liposome control were administered at day 5, 8, and 11 (n=5 per group). At the endpoint (day 23), mice were sacrificed for further analysis. For survival experiments, mice were closely monitored until the humane euthanization endpoint (day 40). (FIG. 14B) Individual tumor growth kinetics of the different experimental groups. (FIG. 14C) Primary injection-site tumor sizes of the different experimental groups at the endpoint (day 23). (FIG. 14D) Quantification of primary injection-site tumor sizes of the different experimental groups at the endpoint (day 23). An unpaired t-test was used with multiple t-test adjustments. Data are expressed as mean±SEM. (FIG. 14E) Survival analysis of experimental groups (n=5). The Mantel-Cox test was used. *p<0.05, ns refers to “not significant,” Immunofluorescence staining and grading of TLSs and immune cell densities in the drug-encapsulated HA-CPP⊂BII[8]hydrogel-treated CT26 tumors (F—K). (FIG. 14F) Formation of TLSs in the CT26 tumor of different experimental groups: Hydrogel-LIGHT+CXCL13 mRNA (scale bar=200 μm), LIGHT+CXCL13 mRNA (scale bar=100 μm), Control Hydrogel only (scale bar=100 μm), T-cells (CD3 marker), B-cells (B220 marker) and antigen-presenting cells (CD11c) were displayed in the tumor microenvironment of the different experimental groups. (FIG. 14G) Quantification of TLSs number per mm² in each experimental cohort. Kolmogorov Smirnoff test was used to assess the statistical difference. (FIG. 14-1) Formation of mature germinal center-like (Ki67+) TLS in the Hydrogel-LIGHT+CXCL13 mRNA group (scale bar=100 μm) and immature TLS in the LIGHT+CXCL13 mRNA group (scale bar=100 μm). (FIG. 14I) TLSs grading (mature, immature, no TLS) after immunostaining of treated CT26 tumors (n=5 per group). Chi-square contingency analysis was performed. (FIG. 14J) Quantification of B220+ B cells per mm² from the tumors of various experimental groups. (FIG. 14K) Quantification of CD3+ T cells per mm² from the tumors of various experimental groups. An unpaired t-test with Welch correction was used for statistical analysis. Data were expressed as mean±SEM. *p<0.05, **p<0.01, ***p<0.001, ****p<0.0001, ns refers to “not significant”.

FIGS. 15A-15I. In vivo delivery of CXCL13 and LIGHT mRNA by HA-CPP⊂CB[8]hydrogel in combination with anti-PD1 therapy into 4T1 tumor-bearing mice. (FIG. 15A) Treatment regimen of HA-CPP⊂CB[8]hydrogel-loaded with liposome-encapsulated CXCL13 and LIGHT mRNA in combination with anti-PD1 therapy in 4T1 mice model. Mice were inoculated with 4T1 breast cancer cell line by subcutaneous injection at day 0. Intratumoral injections of drug-encapsulated hydrogel (Hydrogel-LIGHT+CXCL13 mRNA), drugs alone (LIGHT+CXCL13 mRNA), vehicle hydrogel control or vehicle liposome control were administered at day 5, 8, and 11 (n=5 per group), Anti-PD1 treatment was intravenously administered in combination or alone at days 15 and 17. At the endpoint (day 23), mice were sacrificed for further analysis. For survival experiments, mice were closely monitored until the humane euthanization endpoint (day 40), (FIG. 15B) Individual tumor growth kinetics of the different experimental groups. (FIG. 15C) Primary injection-site tumor sizes of the different experimental groups at the endpoint (day 23). (FIG. 15D) Quantification of primary injection-site tumor sizes of the different experimental groups at the endpoint (day 23). An unpaired t-test was used with multiple t-test adjustments. Data are expressed as mean±SEM. (FIG. 15E) Survival analysis of experimental groups (n=5). The Mantel-Cox test was used. *p<0.05, ns refers to “not significant.” Immunofluorescence staining and grading of TLSs and immune cell densities in the drug-encapsulated HA-CPP⊂CB[8]hydrogel-treated 4T1 tumors (F—K). (FIG. 15F) Formation of TLSs in the 4T1 tumor of different experimental groups: Hydrogel-LIGHT+CXCL13 mRNA+anti-PD1 (scale bar=200 μm), Hydrogel-LIGHT+CXCL13 mRNA (scale bar=100 μm), Control anti-PD1 only (scale bar=100 μm). T-cells (CD3 marker), B-cells (B220 marker) and antigen-presenting cells (CD11c) were displayed in the tumor microenvironment of the different experimental groups. (FIG. 15G) Quantification of TLSs number per mm² in each experimental cohort. Kolmogorov Smirnoff test was used to assess the statistical difference, (FIG. 151H) Formation of mature germinal center-like (Ki67+) TLS in the Hydrogel-LIGHT+CXCL13 mRNA group (scale bar=100 μm) and immature TLS in the LIGHT+CXCL13 mRNA group (scale bar=100 μm). (FIG. 15I) TLSs grading (mature, immature, no TLS) after immunostaining of treated 4T1 tumors (n=5 per group). Chi-square contingency analysis was performed.

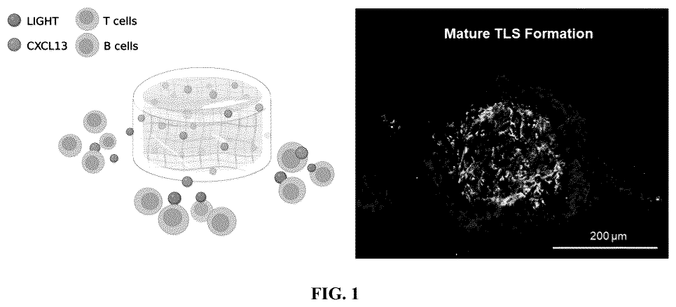

### 4.1 DEFINITIONS

The term “treatment” as used herein are intended to mean obtaining a desired pharmacological and/or physiologic effect, e.g., inhibiting cancer growth or ameliorating ischemic injury to an organ (e.g., heart). The effect may be prophylactic in terms of completely or partially preventing a disease or symptom thereof and/or therapeutic in terms of a partial or complete cure for a disease and/or adverse effect attributable to the disease. “Treatment” as used herein includes preventative (e.g., prophylactic), curative or palliative treatment of a disease in a mammal, particularly human; and includes: (1) preventative (e.g., prophylactic), curative or palliative treatment of a disease or condition (e.g., a cancer or ischemia disease) from occurring in an individual who may be pre-disposed to the disease but has not yet been diagnosed as having it; (2) inhibiting a disease (e.g., by arresting its development); or (3) relieving a disease (e.g., reducing symptoms associated with the disease).

The term “administered”, “administering” or “administration” are used interchangeably herein to refer a mode of delivery, including, without limitation, oral, nasal, pulmonary, transdermal, such as passive or iontophoretic delivery, or parenteral, e.g., rectal, depot, subcutaneous, intravenous, intramuscular, intraperitoneally, intraarterially, intra-cerebella, ophthalmic solution or an ointment.

The term “an effective amount” as used herein refers to an amount effective, at dosages, and for periods of time necessary, to achieve the desired result with respect to the treatment of a disease. For example, in the treatment of a cancer, an agent (i.e., a compound or a composition) which decrease, prevents, delays or suppresses or arrests any symptoms of the cancer would be effective. An effective amount of an agent is not required to cure a disease or condition but will provide a treatment for a disease or condition such that the onset of the disease or condition is delayed, hindered or prevented, or the disease or condition symptoms are ameliorated. The effective amount may be divided into one, two or more doses in a suitable form to be administered at one, two or more times throughout a designated time period.

The term “subject” or “patient” refers to an animal including the human species that is treatable with the method of the present invention. Accordingly, the term “subject” or “patient” comprises any mammal which may benefit from the treatment method of the present disclosure. The term “subject” includes, but is not limited to, mammals. Non-limiting examples include rodents, aquatic mammals, domestic animals such as dogs and cats, farm animals such as sheep, pigs, cows and horses, and humans.

The term “cancer” as used herein is intended to mean any cellular malignancy whose unique trait is the loss of normal controls that results in unregulated growth, lack of differentiation and ability to invade local tissues and metastasize. Cancer can develop in any organ or tissue, and may be any of breast cancer, cervical cancer, ovary cancer, endometrial cancer, melanoma, uveal melanoma, brain tumor, lung cancer, liver cancer, lymphoma, neuroepithelioma, kidney cancer, bladder cancer, pancreatic cancer, prostate cancer, stomach cancer, colon cancer, uterus cancer, hematopoietic tumors of lymphoid lineage, myeloid leukemia, thyroid cancer, thyroid follicular cancer, myelodysplastic syndrome (MDS), tumor of mesenchymal origin, teratcarcinoma, neuroblastoma, glioma, glioblastoma, keratoacanthomas, analplastic large cell lymphoma, esophageal squamous cell carcinoma, follicular dentritic cell carcinoma, intestinal cancer, muscle invasive cancer, seminal vesicle tumor, epidermal carcinoma, spleen cancer, head and neck cancer, stomach cancer, bone cancer, cancer of retina, biliary cancer, small bowel cancer, salivary gland cancer, uterine sarcoma, cancer of testicles, cancer of connective tissue, prostatic hypertrophy, myelodysplasia, Waldenstrom's macroglobulinemia, nasopharyngeal, neuroendocrine cancer, mesothelioma, angiosarcoma, Kaposi's sarcoma, oesophagogastric, fallopian tube cancer, peritoneal cancer, papillary serous mullerian cancer, malignant ascites, gastrointestinal stromal tumor (GIST), Li-Fraumeni syndrome or Von Hippel-Lindau syndrome (VHL). The hematopoietic tumors of lymphoid lineage may be any of leukemia, acute lymphocytic leukemia, chronic lymphocytic leukemia, B-cell lymphoma, Burkitt's lymphoma, multiple myeloma, Hodgkin's lymphoma, or Non-Hodgkin's lymphoma. The myeloid leukemia may be acute myelogenous leukemia (AML) or chronic myelogenous leukemia (CML). The tumor of mesenchymal origin is fibrosarcomas or rhabdomyosarcomas.

### 5. DETAILED DESCRIPTION

Tertiary lymphoid structures (TLSs) within the tumor microenvironment have been correlated with favorable cancer prognosis and enhanced immune checkpoint blockade (ICB) responses. The inventors engineered an injectable hydrogel-based drug formulation to stimulate TLS formation in a B16 melanoma mouse model. The hydrogel, termed HA-CPP⊂CB[8], was formed via supramolecular interactions between 4-(4-chlorophenyl)pyridine modified hyaluronic acid (HA-CPP) and cucurbit[8]uril (CB[8]). A single injection of HA-CPP⊂CB[8]hydrogel encapsulating the CXCL13 chemokine and LIGHT cytokine effectively suppressed tumor growth, prolonged survival, increased TLS density, and promoted TLS maturation. Notably, the hydrogel intervention also up-regulated OVA antigen-spreading in the secondary lymphoid organs. Moreover, combining the hydrogel-based drug formulation with the anti-PD1 ICB therapy led to augment tumor suppression, improve survival rates, and enhanced TLS formation, contributing to B16 melanoma eradication. Provided is hydrogel-based drug carriers and methods of using the hydrogel-based drug carriers as synthetic immune niches for stimulating mature-like TLS formation within the B16 melanoma tumor microenvironment.

### 5.1 COMPOUNDS

In some embodiments, the present disclosure provides a compound comprising hyaluronic acid and 4-(4-chlorophenyl)pyridine (HA-CPP) or a salt thereof. In some embodiments, the compound is a conjugate of hyaluronic acid (HA) and 4-(4-chlorophenyl)pyridine (CPP) wherein the HA and CPP are covalently linked. In some embodiments, the HA and CPP are linked directly. In some embodiments, the HA and CPP are linked through a linker.

Hyaluronic acid (HA) is a linear polysaccharide with a high molecular weight that is made up of repeating disaccharides of D-glucuronic acid and N-acetyl-D-glucosamine. These disaccharides are linked by a glucuronidic β (1→3) bond. The compounds of the present disclosure comprise at least one 4-(4-chlorophenyl)pyridine (CPP) linked to HA.

In some embodiments, a linker is used to connect the CPP to the HA. The linker can be any moiety having a functional group that can form a bond with one or more functional groups on HA. In some embodiments, the linker is an alkylenamine. CPP can react with an alkylamine derivative containing a leaving group to form an amine derivative of CPP, for example CPP—(CH₂)ₘ—NH₂ wherein m is an integer, for example 1-10, e.g., 1, 2, 3, 4, 5, 6, 7, 8, 9 or 10. The amine group can then be linked to HA, for example by forming a bond with one or more carboxylic acid group of a D-glucoronic acid subunit. In some embodiments, the linker is —(CH₂)₃—NH—.

The degree of substitution of CPP on the HA scaffold (i.e., how many disaccharide subunits are substituted with CPP) can vary. In some embodiments, about 10-30% of hyaluronic acid disaccharide subunits are substituted with CPP (i.e., the degree of substitution is about 0.1-0.3). In some embodiments, about 15% of hyaluronic acid disaccharide subunits are substituted with CPP (i.e., the degree of substitution is about 0.15). In some embodiments, the degree of substitution of CPP on the HA scaffold is about 0.05, about 0.1, about 0.15, about 0.2, about 0.25, about 0.3, or higher.

In some embodiments, HA-CPP comprises at least one moiety of formula (1):

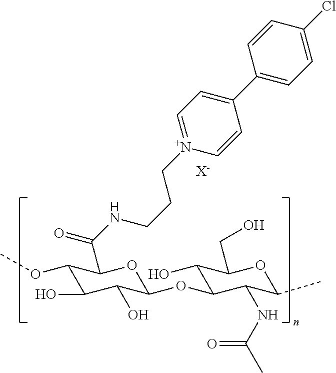

wherein n is an integer and X is a counter-ion;
or a salt thereof.

The integer can be any number between 1 and xx [INVENTORS PLEASE COMPLETE]

In some embodiments, the compound comprises repeating units of the compound of formula (1) and unsubstituted HA

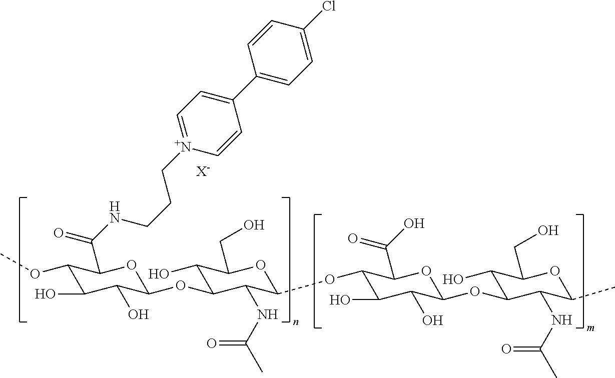

wherein each of n and m are each independently at each occurrence integer of 1 to [[xx.]]

In some embodiments, the overall ratio of n to m is about 0.1. In some embodiments, the overall ratio of n to m is about 0.15. In some embodiments, the overall ratio of n to m is about 0.2. In some embodiments, the overall ratio of n to m is about 0.25. In some embodiments, the overall ratio of n to m is about 0.3.

In some embodiments, the HA-CPP comprises at least one moiety of formula (1a):

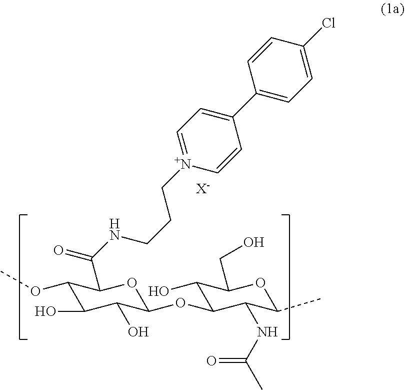

In some embodiments, the HA-CPP comprises between about 5% to about 50% of the moiety of formula (1a). In some embodiments, the HA-CPP comprises between about 10% to about 30% of the moiety of formula (1a). In some embodiments, the HA-CPP comprises between about 15% of the moiety of formula (1a). In some embodiments, the HA-CPP comprises between about 20% of the moiety of formula (1a). In some embodiments, the HA-CPP comprises between about 25% of the moiety of formula (1a). In some embodiments, the HA-CPP comprises between about 30% of the moiety of formula (1a).

In some embodiments, the present disclosure provides a composition comprising (a) a compound comprising hyaluronic acid and 4-(4-chlorophenyl)pyridine (HA-CPP) or a salt thereof and a cucurbit[n]uril (CB[n]), wherein n is 5, 6, 7 or 8 (HA-CPP⊂CB[n]).

In some embodiments, n is 5 (HA-CPP⊂CB[5]. In some embodiments, n is 6 (HA-CPP⊂CB[6]). In some embodiments, n is 7 (HA-CPP⊂CB[7]). In some embodiments, n is 8 (HA-CPP⊂CB[8]).

In some embodiments, the composition is in the form of a hydrogel.

In some embodiments, the composition is an injectable composition and comprises water at a weight ratio of about 1-5%.

In some embodiments, the HA-CPP and CBI[n] are linked through non-covalent binding.

In some embodiments, the composition further comprises one or more biologically active agents. The HA-CPP⊂CB[n] can be combined with the biologically active agent through non-covalent binding to form a complex. In some embodiments, HA-CPP⊂CB[8] can be combined with the biologically active agent through non-covalent binding to form a complex.

Any biologically active agent can be utilized in the compositions of the present disclosure. In some embodiments, the biologically active agent is a chemokine, cytokine, immune-checkpoint inhibitor, cancer vaccine, chimeric antigen receptor or a combination thereof. In some embodiments, the biologically active agent is CXCL13, CCL21, IL-4, IL-7, IL-2, LIGHT, CCL19, CXCL12, CXCL13, lymphotoxin, TNF-α or a combination thereof. In some embodiments, the chemokine is CXCL13 and the cytokine is LIGHT.

### 5.2 METHODS OF PREPARATION

In some embodiments, the present disclosure provides a method of making HA-CPP comprising: (a) combining 4-(4-chlorophenyl)pyridine with a compound of the formula X—(CH₂)₃—NH₂ wherein X is a leaving group; and (b) reacting the product of step (a) with hyaluronic acid (HA). In some embodiments, the HA has a molecular weight of about 6 kDa to 1000 kDa. In some embodiments, the leaving group is a halogen (e.g., F, Cl, Br, I). In some embodiments, the leaving group is a sulfonate (e.g., an alkylsulfonate or an arylsulfonate).

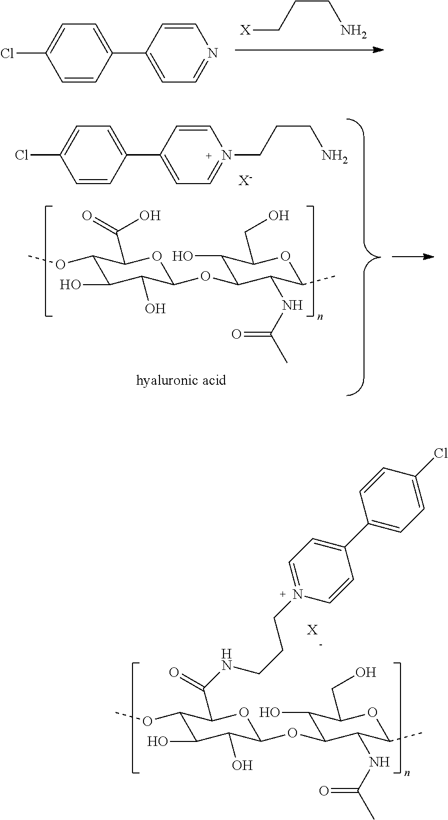

In some embodiments, the present disclosure provides a method of making HA-CPP comprising: (i) combining 4-(4-chlorophenyl)pyridine with a 3-halopropylamine to form a pyridinium salt CPP—NH₂ and (ii) performing an amidation reaction with at least one glucuronic acid subunit of hyaluronic acid (HA). In some embodiments, the HA has a molecular weight of about 6 kDa to 1000 kDa.

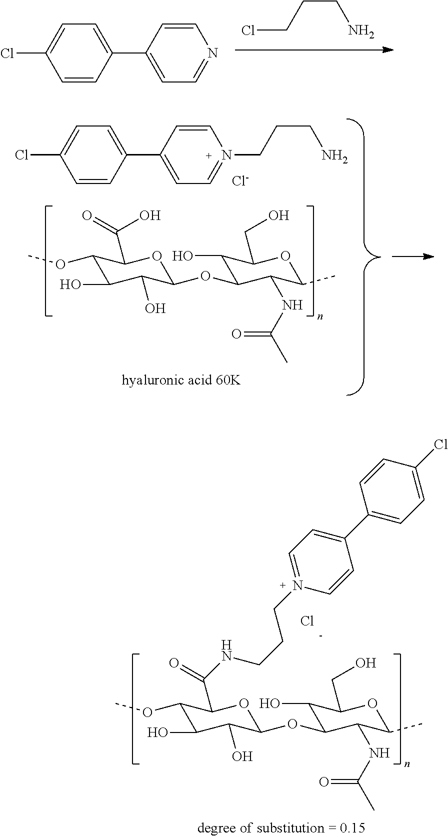

In some embodiments, the method further comprises combining HA-CPP and cucurbit[n]uril (CBI[n]) wherein n is 5, 6, 7 or 8 to form HA-CPP⊂CB[n]. In some embodiments, n is 8 and the method comprises combining HA-CPP and cucurbit[8]uril (CB[8]) to form HA-CPP⊂CB[8].

In some embodiments, wherein the stoichiometric ratio between CPP and CB[8] is 2:1. In some embodiments, the stoichiometric ratio between CPP and CB[8] is greater than 2:1.

### 5.3 FORMULATION

The compound of the present disclosure may be formulated into liquid pharmaceutical compositions, which are sterile solutions, or suspensions that can be administered by, for example, intravenous, intramuscular, subcutaneous, or intraperitoneal injection; whereas the composition may be in any dosage form, which includes without limiting, solid or liquid dosage for oral, parenteral, nasal or sublingual administration. Suitable diluents or solvent for manufacturing sterile injectable solution or suspension of the mixture of the present disclosure include, but are not limited to, 1,3-butanediol, mannitol, water, Ringer's solution, and isotonic sodium chloride solution. Fatty acids, such as oleic acid and its glyceride derivatives are also useful for preparing injectables, as are natural pharmaceutically-acceptable oils, such as olive oil or castor oil. These oil solutions or suspensions may also contain alcohol diluent or carboxymethyl cellulose or similar dispersing agents. Other commonly used surfactants such as Tweens or Spans or other similar emulsifying agents or bioavailability enhancers that are commonly used in manufacturing pharmaceutically acceptable dosage forms can also be used for the purpose of formulation. Oral administration may be either liquid or solid composition form.

The composition is administered to a subject to elicit a desired response, factors such as disease state or severity of the condition to be alleviated, age, sex, weight of the patient, the state of being of the subject, and the severity of the pathological condition being treated, concurrent medication or special diets then being followed by the subject, and other factors which those skilled in the art will recognize, with the appropriate dosage ultimately being at the discretion of the attendant physician. Dosage regimens may be adjusted to provide the desired response. The composition of the present disclosure is administered at an amount and for a time such that at least one dosages of the compound (e.g., 2, 3, 4 or even more dosages) is administered to the subject to achieve an improved therapeutic response.

### 6 EXAMPLES

### 6.1 Development and Characterization of HA-CPP⊂CB[8] Hydrogel-Based Drug Delivery Systems

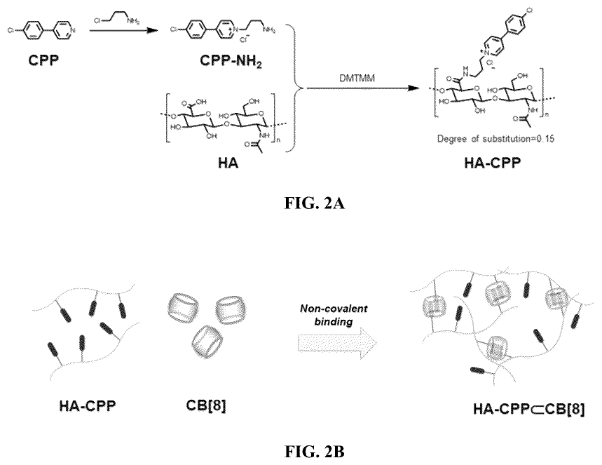

We developed an injectable hyaluronic acid (HA)-hydrogel-based drug-delivery system for CXCL13 and LIGHT proteins. To synthesize the HA polymer modified with 4-(4-chlorophenyl)pyridine (HA-CPP), we followed a well-defined synthesis route (FIG. 2A). Initially, we combined CPP and 3-Chloropropylamine, resulting in the formation of a pyridinium salt called CPP—NH2. Then, we performed an amination reaction with HA (molecular weight (MW) of 60K) to successfully generate the desired HA-CPP (FIG. 2B).

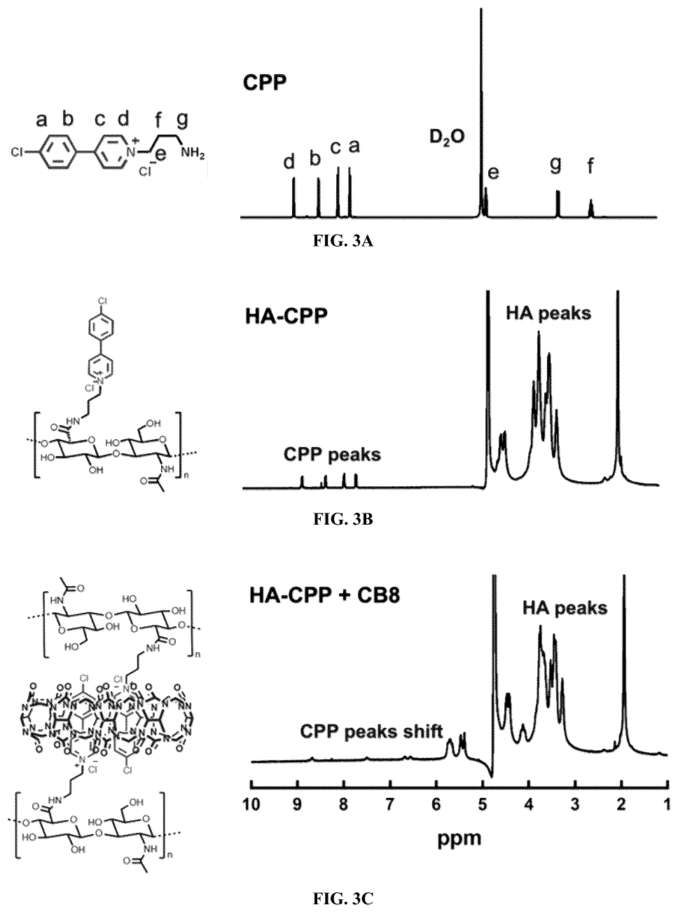

To determine the average degree of CPP substitution, we integrated the peaks in the NMR spectrum, yielding a calculated value of 0.15. We then mixed HA-CPP with CB [8] at a stoichiometric ratio of 2:1 CPP:CB[8] to form a complex, namely HA-CPP⊂CB[8](Scheme 1B). This complexation was confirmed through 1H-NMR, which exhibited a noticeable shift in the spectra of the aromatic protons of CPP, indicating the successful insertion of pendant CPP guests within the CB[8] cavity (FIGS. 3A-3C).

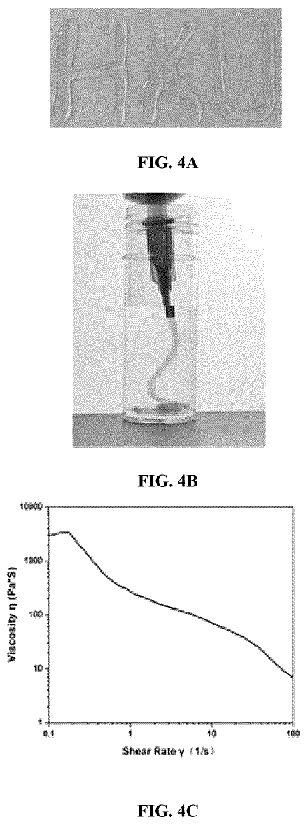

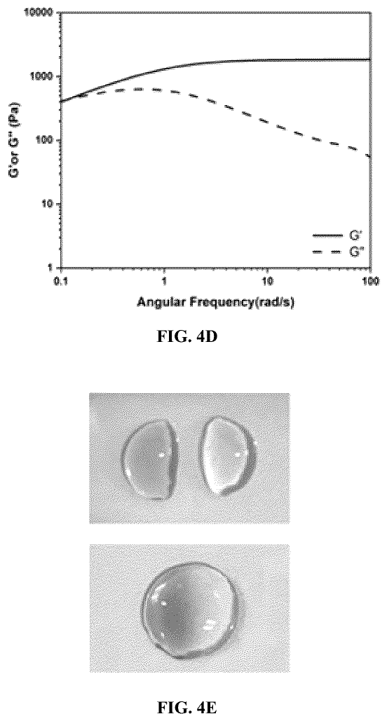

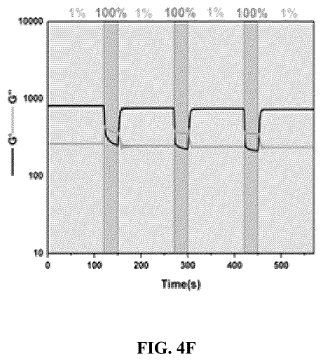

We further formed the HA-CPP⊂CB[8]hydrogel by dissolving the lyophilized complexation in deionized (DI) water at a weight ratio of 2%. The 2% HA-CPP⊂CB[8]hydrogel displayed ideal injectability, as it smoothly passed through the syringe needle via shear thinning and maintained its shape after the shear force disappeared (FIGS. 4A and 4B). The HA-CPP⊂CB[8]hydrogel also presents shear-thinning properties in the flow ramp experiment, which further demonstrates its injectability (FIG. 4C).

We performed rheological tests to examine the dynamic viscoelastic behavior of networks created from CPP⊂CB[8] supramolecular crosslinks. Samples were first subjected to an amplitude sweep to determine the linear viscoelastic range and then tested by the oscillatory frequency sweep in this range. The critical frequency (ω_c) where G′=G″ was close to the lowest tested angular frequency (0.1 rad/s), indicating a hydrogel formed with G′>G″ in the whole tested frequency range (FIG. 4D). Furthermore, the HA-CPP⊂CB[8]hydrogel exhibited self-healing properties due to the reversible host-guest complexation between the polymer side chains. We demonstrated this by joining two differently stained hydrogels and observing the self-healing effect optically (FIG. 4E). We also performed cyclic step-strain measurements on the HA-CPP⊂CB[8]hydrogel under minor (1%) and major (100%) strains to validate the self-healing capabilities of this supramolecular hydrogel. The hydrogel formed a self-supporting structure at 1% strain, with G′ exceeding G″, but transitioned into a sol state at 100% strain, with G′ and G″ values switching (FIG. 4F). This pattern repeated in subsequent cycles of step strain. Leveraging from the dynamic, the injectability and self-healing properties of the hydrogel warrant the application of the hydrogel to be injected near tumors and maintain its shape in the location.

### 6.2 In Vivo HA-CPP⊂CB[8] Hydrogel-Based Delivery of CXCL13 and LIGHT Presents Anti-Tumor Effects in the Intraperitoneal B16-OVA Mice Model

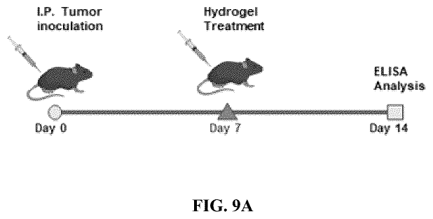

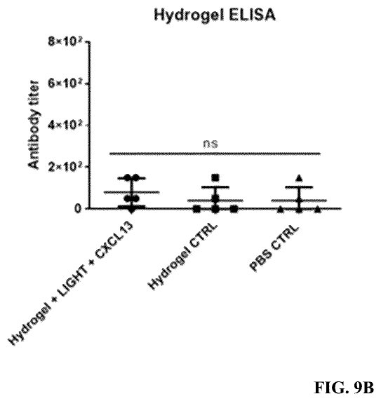

We first injected blank hydrogel without encapsulating drugs (control hydrogel) intraperitoneally into female C57 mice to assess potential in vivo toxicities. Mice injected with the control hydrogel showed no signs of discomfort or weight loss after the injection. Serum ELISA results obtained at Day 14 showed no immunogenic reactions related to the HA-CPP⊂CB[8]hydrogel (FIGS. 9A-9B).

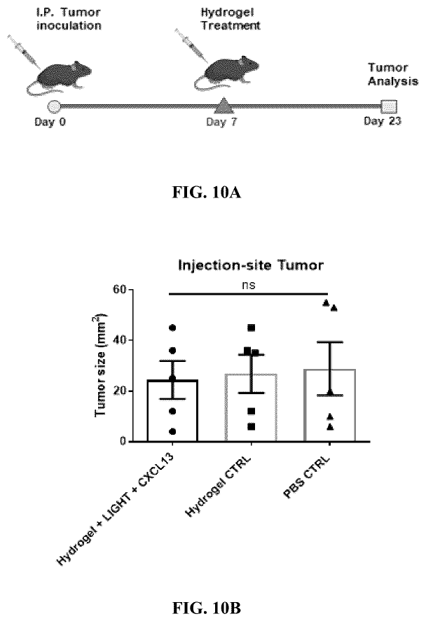

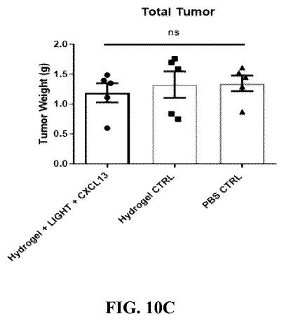

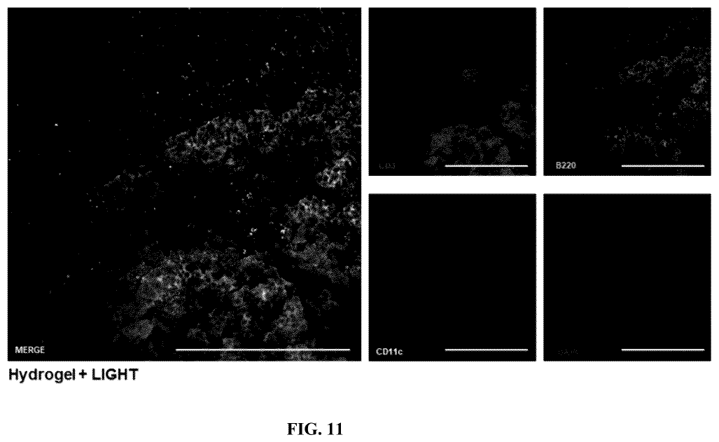

Our preliminary study first immunized intraperitoneally challenged B16 tumor-bearing mice with a LIGHT-encapsulated hydrogel formulation (FIGS. 10A-10C). Enhanced T cell cluster recruitment was uniquely observed in the LIGHT-encapsulated hydrogel group (FIG. 11). However, there were no observable changes in primary tumor sizes or total tumor weight by the endpoint (FIGS. 10A-0C). This result suggests that only T cell recruitment by LIGHT might not be sufficient for anti-tumor activity. Therefore, we combined LIGHT (T cell recruitment factor) and CXCL13 (B cell recruitment factor) for the final drug-encapsulated hydrogel formulation 12.

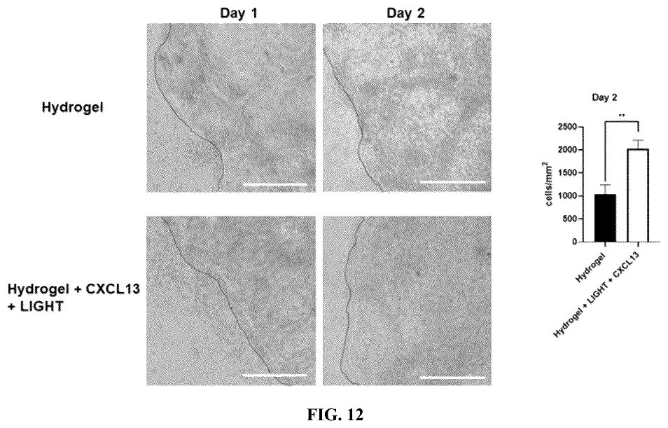

We found that HA-CPP⊂CB[8]hydrogels loaded with CXCL13 and LIGHT proteins significantly recruit more immune cells than the control hydrogel alone under in vitro culture conditions (FIG. 12).

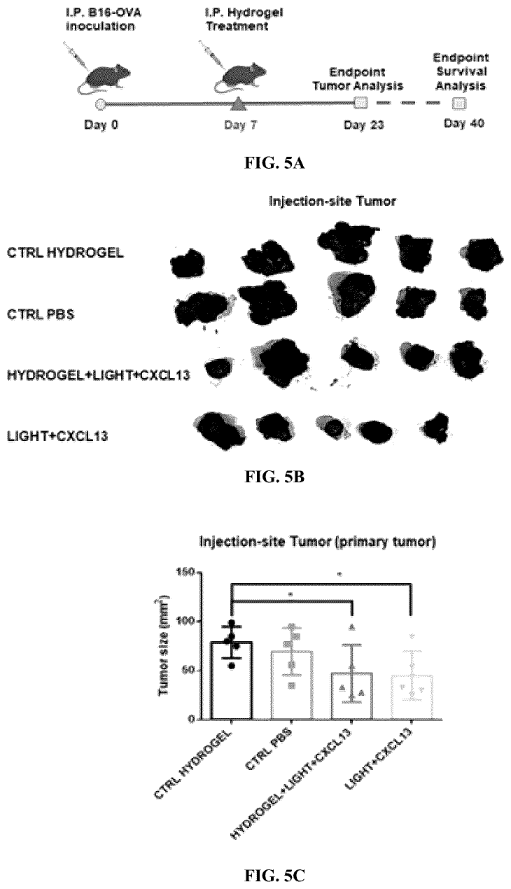

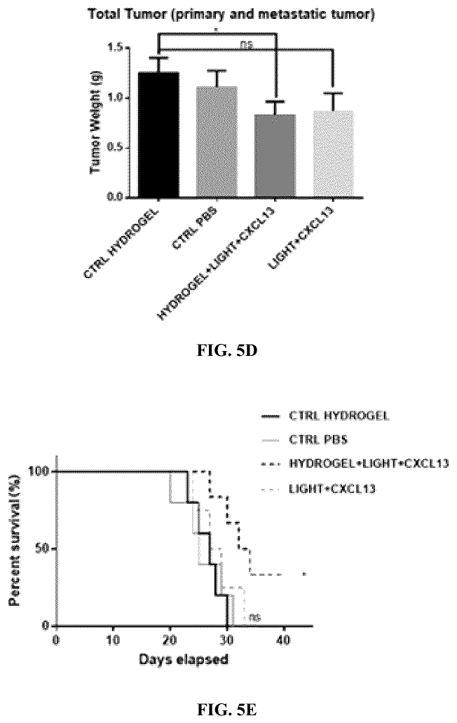

In our subsequent experiment, we injected randomized female C57 mice intraperitoneally with B16-OVA melanoma (3×10⁵ cells/animal) on day 0, followed by injections of drug-encapsulated hydrogel formulation (hydrogel-delivered 0.6 μg CXCL13+0.6 μg LIGHT), drugs-alone (0.6 μg CXCL13+0.6 μg LIGHT in PBS), vehicle-hydrogel control, or PBS control on day 7 at the same site (FIG. 5A). We observed no signs of discomfort among the mice in the different experimental groups. At the endpoint (day 23), all mice were sacrificed, and their tumors and spleens were surgically removed for further immunological analysis. Interestingly, both the drug-encapsulated hydrogel formulation and drugs-alone groups exhibited partial primary tumor suppression (FIGS. 5B and 5C). When comparing injection-site tumor sizes (primary intraperitoneal tumor) with vehicle-hydrogel control and PBS control tumors (FIG. 5B), both the drug-encapsulated hydrogel formulation and drugs-alone groups formed significantly smaller B16-OVA tumors (average tumor sizes of 47.2 mm2 and 45.7 mm2, respectively, compared to the vehicle-hydrogel control group's average tumor size of 78.8 mm2) (FIG. 5C).

We also quantified the total tumor weights (primary and metastatic intraperitoneal tumors) from all the groups. Our results revealed that only the drug-encapsulated hydrogel formulation group led to significant tumor weight reductions (average tumor weight of 0.83 g) when compared with the vehicle control (average tumor weight of 1.25 g) (FIG. 5D). This outcome may be attributed to the enhanced recruitment of cancer-suppressing immune cell infiltrations generated by the hydrogel's matrix³⁵. Notably, the drug-encapsulated hydrogel formulation group also significantly prolonged mouse survival (1/5 death-free mice by the day 40 experimental endpoint) compared to the control groups (FIG. 5E). This outcome may also be attributed to the enhanced recruitment of cancer-suppressing immune cell infiltrations generated by the hydrogel's matrix³⁵. Consequently, we conducted further studies to analyze the immunological niche within the tumor microenvironments and secondary lymphoid organs of the different experimental groups.

### 6.3 In Vivo HA-CPP⊂CB[8] Hydrogel-Based Delivery of CXCL13 and LIGHT Generates Mature TLSs within the B16 Tumor Microenvironment

Using the frozen tumor specimens from the experimental groups previously mentioned (harvested at the day 23 endpoint) (FIGS. 5B-5E), we conducted immunohistochemical staining to reveal the presence of the T cell (CD3 surface marker), B cell (B220 surface marker), and antigen-presenting cell (CD11c surface marker) infiltrations within the tumor microenvironment.

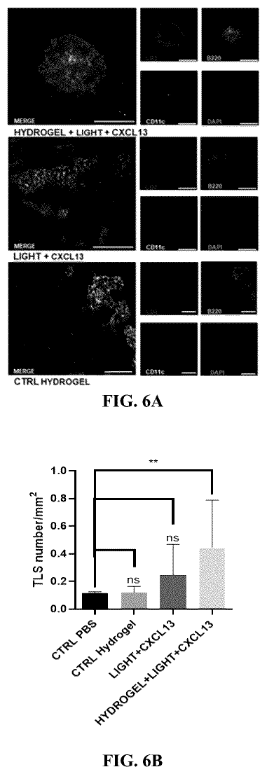

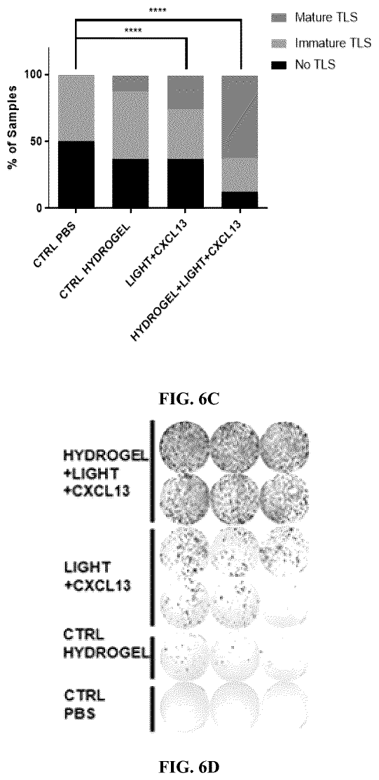

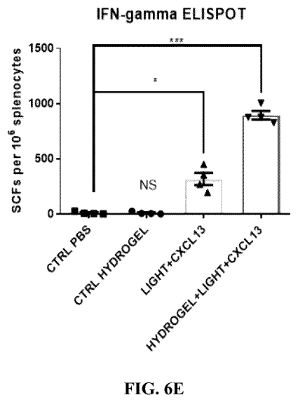

We characterized mature TLSs as densely-packed germinal center-like B cell clusters with surrounding T cell clusters and adjacent antigen-presenting dendritic cells. Interestingly, we predominantly observed these mature structures in the tumor microenvironments of the drug-encapsulated hydrogel formulation group (FIGS. 6A and 6C).

Although we observed TLS-like structures in the other experimental groups, most were immature TLSs, characterized by scattered distribution of clustered B cell and T cell populations (FIG. 6A). Notably, the drug-encapsulated hydrogel formulation group generated significantly greater TLS numbers (average TLS number of 0.45 per mm2) than the other groups (FIG. 6B). Significantly larger proportions of TLS-positive samples from the drug-encapsulated hydrogel formulation group exhibited mature TLS structures when compared with the PBS-control group (FIG. 6C). Overall, these immunohistochemical staining results support the efficacy of the drug-encapsulated hydrogel formulation.

### 6.4 In Vivo HA-CPP⊂CB[8] Hydrogel-Based Delivery of CXCL13 and LIGHT Generates Potent OVA-Antigen T Cell Responses in Secondary Lymphoid Organs

To determine whether the intraperitoneally injection of hydrogel-based delivery of CXCL13 and LIGHT could stimulate antigen-spreading responses in secondary lymphoid organs, we harvested spleens adjacent to the injection sites for interferon-gamma ELISPOT detection. Our results revealed that OVA antigen-activated splenocytes from both the drug-encapsulated hydrogel formulation and drugs-alone groups stimulated potent interferon-gamma T cell responses, which play a critical role in tumor elimination (FIGS. 6D and 6E) 36. Notably, OVA-activated splenocytes from the drug-encapsulated hydrogel formulation group (hydrogel-delivered 0.6 μg CXCL13+0.6 μg LIGHT) generated significantly stronger interferon-gamma T cell responses compared to the drugs-alone and PBS-control groups (FIG. 6E). This finding supports the anti-tumor efficacy observed earlier (FIG. 5D).

### 6.5 In Vivo HA-CPP⊂CB[8] Hydrogel-Based Delivery of CXCL13 and LIGHT in Combination with Anti-PD1 ICB Leads to Potent B16-OVA Melanoma Suppression

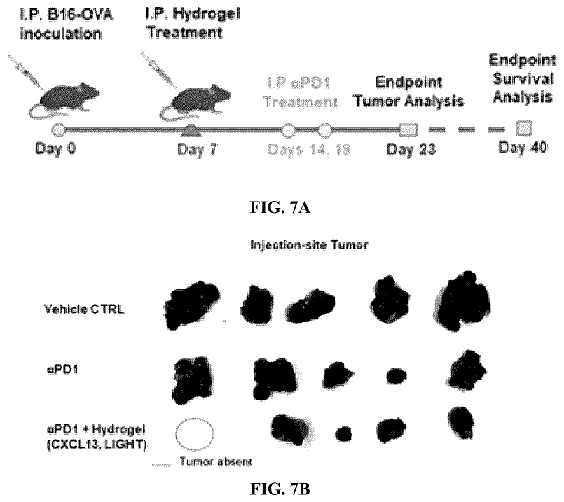

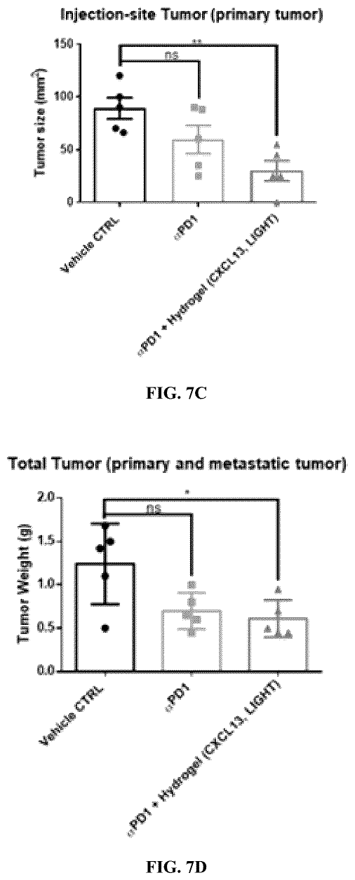

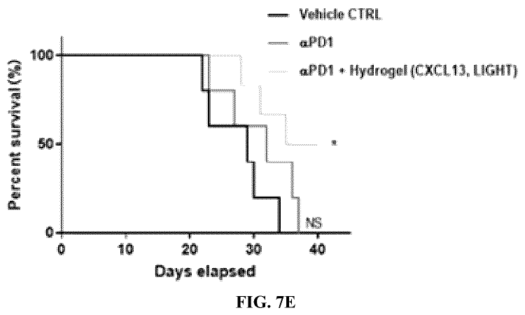

Enhanced formation of TLSs has been previously reported to correlate positively with favorable clinical responses to ICB therapy and improved survival rates in cancer^{12,15}. Therefore, our next experiment aimed to improve B16 tumor eradication by administering a high-dose anti-PD1 (0.6 mg αPD1) drug in combination with the drug-encapsulated hydrogel formulation (hydrogel-delivered 0.6 μg CXCL13+0.6 μg LIGHT) in the intraperitoneal B16 model (FIG. 7A). As anticipated, the combination of drug-encapsulated hydrogel formulation with αPD1 led to the most significant tumor suppression at the endpoint by day 23 (FIGS. 7B-7D). Both the injection-site tumor and total tumor weights were significantly lower in the combination group (average tumor size of 30 mm² and average tumor weight of 0.61 g) compared to the vehicle control group (average tumor size of 89.2 mm2 and average tumor weight of 1.24 g), and the combination group outperformed the αPD1-alone group (FIGS. 7C and 7D). Ultimately, the combination treatment significantly prolonged the survival of B16 tumor-bearing mice (2/5 death-free mice by the day 40 experimental endpoint) compared to the other experimental groups, supporting the therapeutic efficacy of the drugs-encapsulated hydrogel formulation in combination with anti-PD1 (FIG. 7E).

### 6.6 In Vivo HA-CPP⊂CB[8] Hydrogel-Based Delivery of CXCL13 and LIGHT in Combination with Anti-PD1 ICB Generates Mature TLSs within the Tumor Microenvironment and Enhances T Cell Responses in Secondary Lymphoid Organs

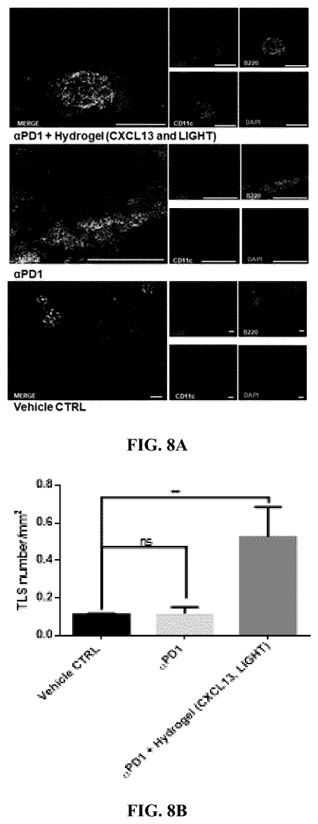

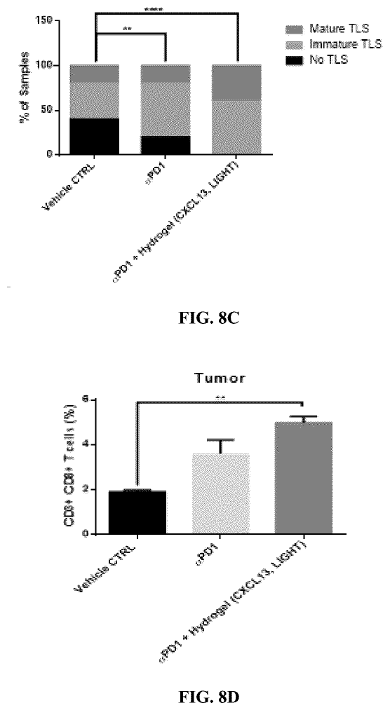

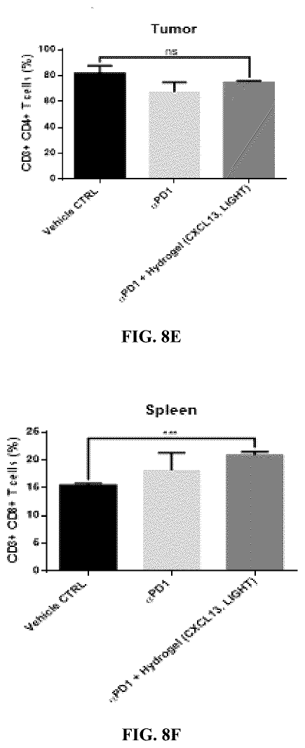

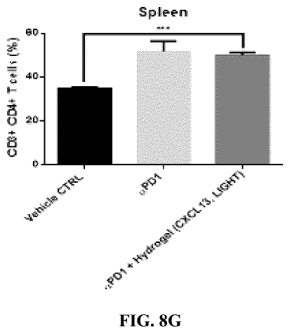

We used the frozen tumor specimens from the experimental groups mentioned earlier (harvested at the day 23 endpoint) to detect the presence of TLSs within B16 tumor microenvironments and grade TLSs maturity (FIGS. 8A-8G). The combination group produced significantly higher TLS numbers in the tumors (average TLS number of 0.524 per mm2) than the αPD1 and vehicle control groups (FIG. 8B). Notably, the mature TLSs were mostly observed in the combination group, while the αPD1 and vehicle control groups primarily exhibited immature TLSs or no TLSs, respectively (FIG. 8C).

We also conducted flow cytometric analysis to explore the potential role of T cell subsets within the tumor microenvironment of the different experimental groups. We observed higher CD8-positive T cell populations in the combination group compared to the vehicle-control group, supporting the cytotoxic role of T cells in eliminating cancer cells within the tumor microenvironment, although the CD4-positive populations did not differ significantly (FIGS. 8D and 8E). In the spleen, the combination treatment induced larger CD8-positive and CD4-positive T cell populations than the vehicle-control (FIGS. 8F and 8G). Overall, these results provide insights into tumor and splenic T cell-based tumoricidal activities generated by the combination treatment.

### 6.7 Hydrogel-Based mRNA Therapeutics for Enhancing Tertiary Lymphoid Structure Formation and Solid Cancer Eradication

In this work, we developed a unique hydrogel formulation to enhance the expression and drug activity of messenger ribonucleic acids (mRNAs). We have performed several experiments to investigate the therapeutic efficacy of our novel hydrogel-based mRNA treatment in various preclinical cancer models (B16.f10 skin cancer, CT26 colon cancer, and 4T1 breast cancer). These experiments highlight the promise of our hydrogel platform in delivering a potent mRNA drug that is effective at treating multiple types of solid cancers. Overall, our results support the enhanced anti-tumor efficacy of our hydrogel-based mRNA treatment compared to the mRNA treatments alone and the vehicle controls. Importantly, we demonstrated that our hydrogel-based mRNA treatment could outcompete the pre-existing anti-PD1 immune checkpoint blockade therapy in suppressing breast cancer, or be administered as a combination therapy against cancer.

### 6.7.1 Skin Cancer Model

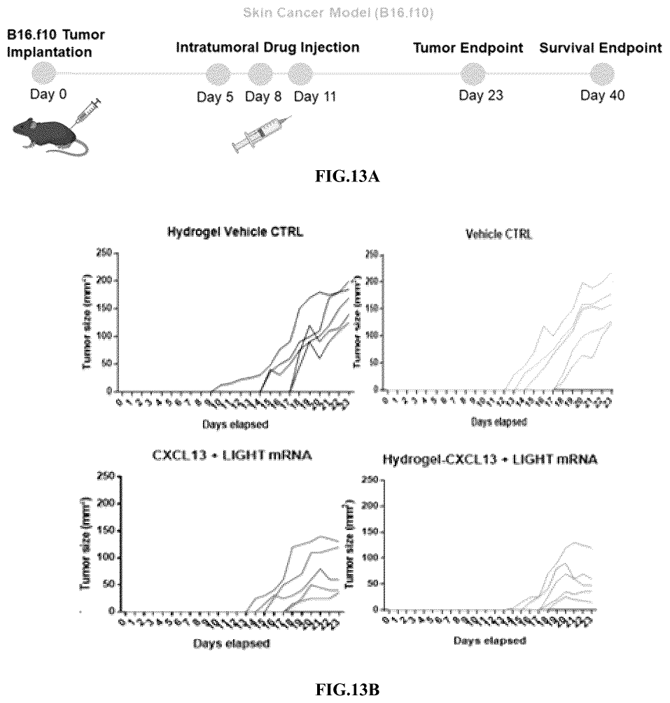

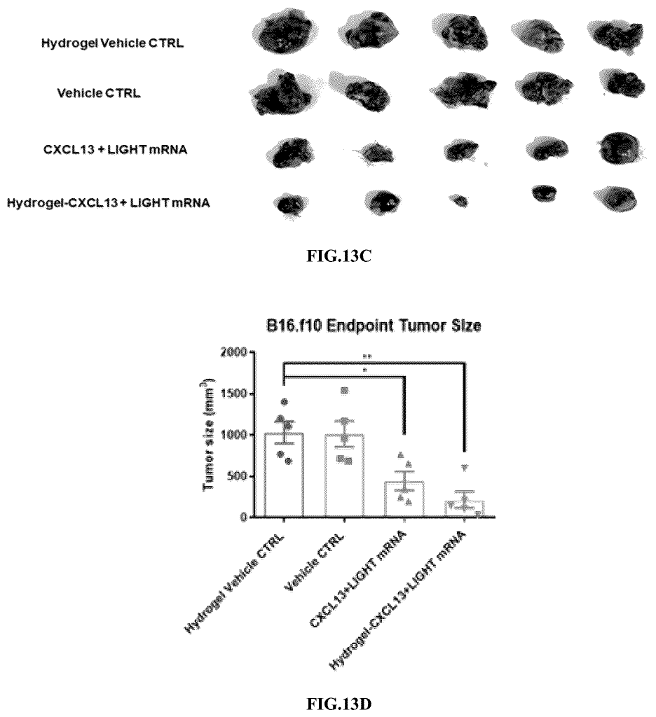

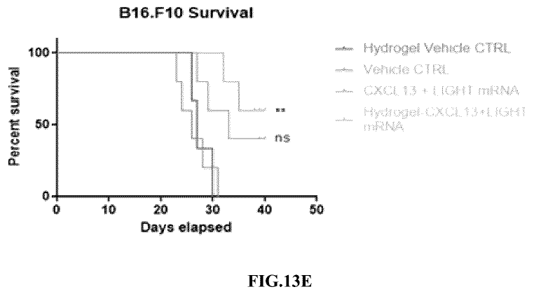

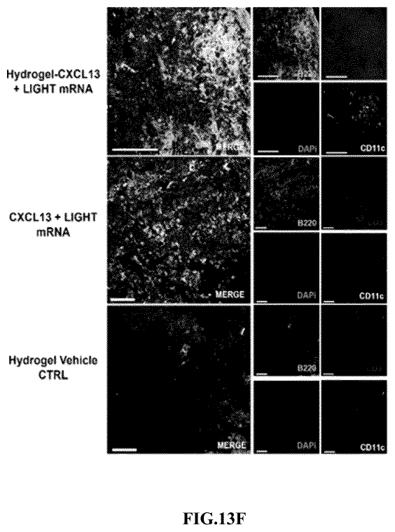

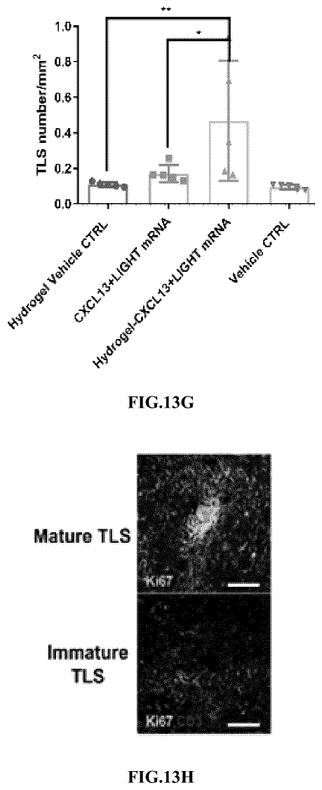

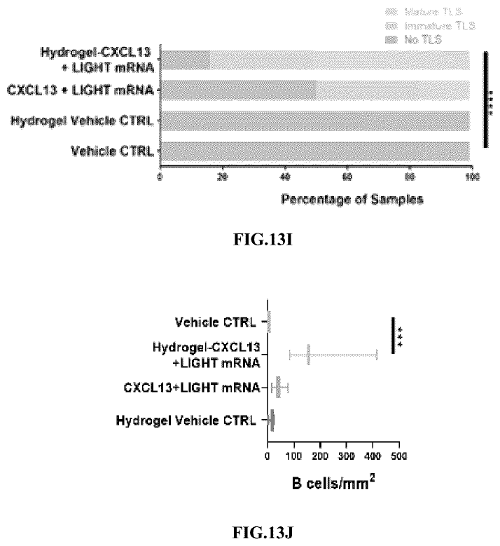

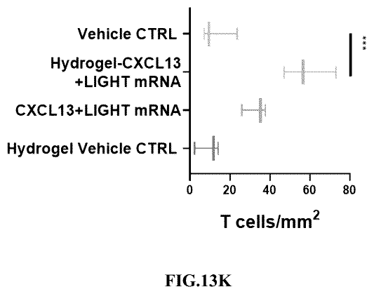

For the skin cancer model, we inoculated female C57BL/6 mice subcutaneously at the right flank with B16.f10 melanoma (2×10⁵ cells/animal) on day 0, followed by injections of the co-delivery hydrogel mRNA formulation (CXCL13 mRNA+LIGHT mRNA in cationic liposome), mRNA drugs alone (CXCL13 mRNA+LIGHT mRNA in cationic liposome), hydrogel control, or vehicle liposome control on days 5, 8, and 11 at the same tumor site (FIG. 13A). We observed no signs of discomfort in mice of the different experimental groups. At the endpoint (day 23), all mice were euthanized, and their tumors were extracted for further analysis. Interestingly, both the co-delivery hydrogel mRNA formulation and co-delivery of mRNA drugs alone exhibited potent primary tumor suppression (FIGS. 13B and 13C). When comparing primary tumor sizes with the hydrogel control and PBS control (FIGS. 13B and 13C), both the co-delivery hydrogel mRNA formulation and co-delivery of the mRNA drugs alone resulted in significantly smaller B16.f10 tumors (average tumor sizes of 218.8 mm³ and 446 mm³, respectively, compared to the hydrogel control group average tumor size of 1032.9 mm³) (FIG. 13D). This result also demonstrated that the co-delivery hydrogel mRNA formulation could better suppress tumor growth than the co-delivery of the mRNA drugs alone. Notably, the co-delivery hydrogel mRNA formulation group also significantly prolonged mouse survival (3/5 of the mice survived by the day 40 experimental endpoint) compared to the control groups (all 5 mice died) (FIG. 13E). This outcome may be attributed to the enhanced recruitment of cancer-suppressing immune cell infiltrations generated by the sustained release of cytokines from the hydrogel⁵². Enhanced TLSs presence in tumors offers a predictor for a strong clinical response to cancer immunotherapies^{53, 54, 55, 56}. Consequently, we conducted further studies to analyze the immunological niche within the tumor of the different experimental groups. Using the frozen tumor specimens from the experimental groups, we conducted immunohistochemical staining and confocal quantification to reveal the presence of TLS by T-cell (CD3 surface marker), B-cell (1B220 surface marker), and antigen-presenting cell (CD11c surface marker) infiltration into the tumors (FIGS. 13F and 13G). We also characterized mature TLSs as densely-packed germinal center-like Ki67-positive cells with surrounding CD3-positive T-cell clusters. Interestingly, we predominantly observed these mature structures in the tumors of the co-delivery hydrogel mRNA formulation group (FIGS. 13H and 13I), Although we observed TLS-like structures in the other experimental groups, most were immature TLSs, characterized by scattered distribution of clustered B-cell, Ki67-positive cell and T-cell populations (FIGS. 13H and 13I). Notably, the co-delivery hydrogel mRNA formulation group generated significantly greater number of TLSs (average TLSs number of 0.47 per mm²) than the other groups (FIG. 13G), Significantly larger proportions of TLSs-positive samples from the co-delivery hydrogel mRNA formulation group exhibited mature TLSs structures when compared with the PBS-control group (FIG. 13G), along with enhanced densities of tumor-infiltrating B cells and T cells (FIGS. 13J and 13K) Overall, immunohistochemical staining results support the efficacy of CXCL13 mRNA and LIGHT mRNA co-delivery using the hydrogel formulation.

### 6.7.2 Colon Cancer Model

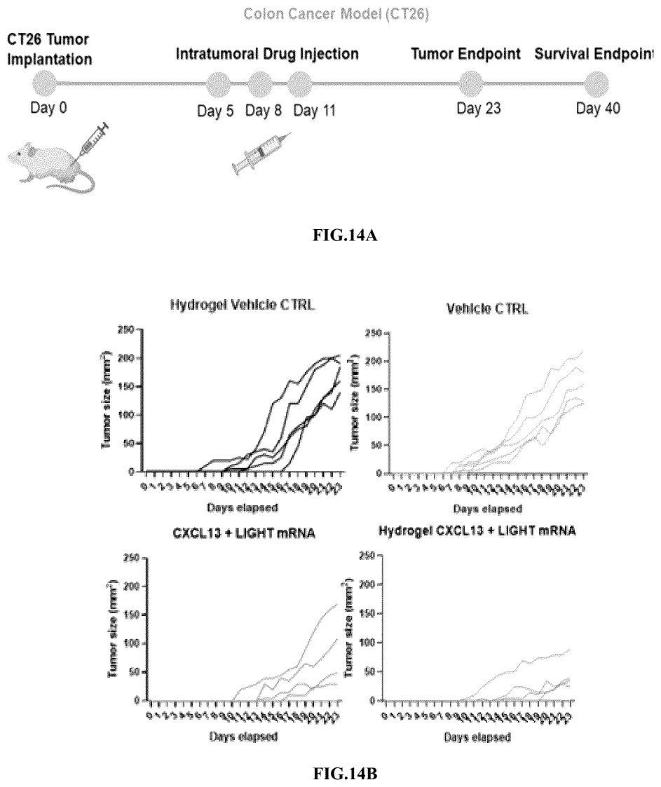

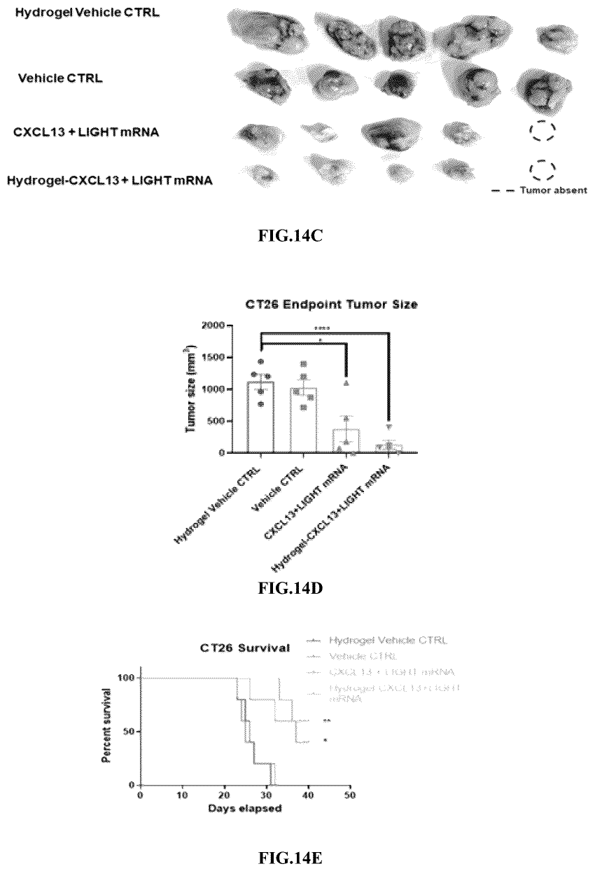

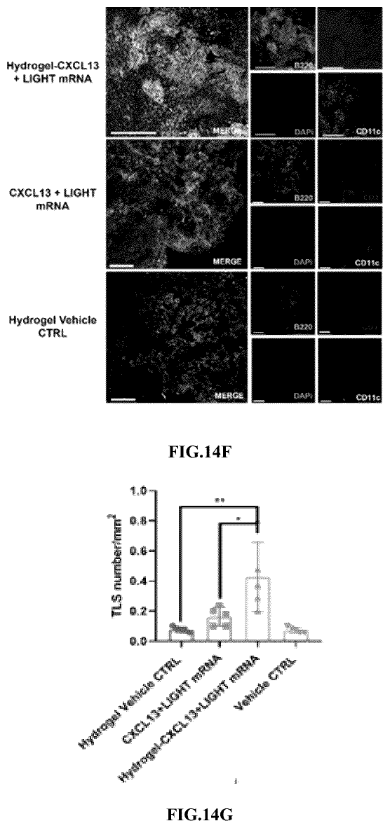

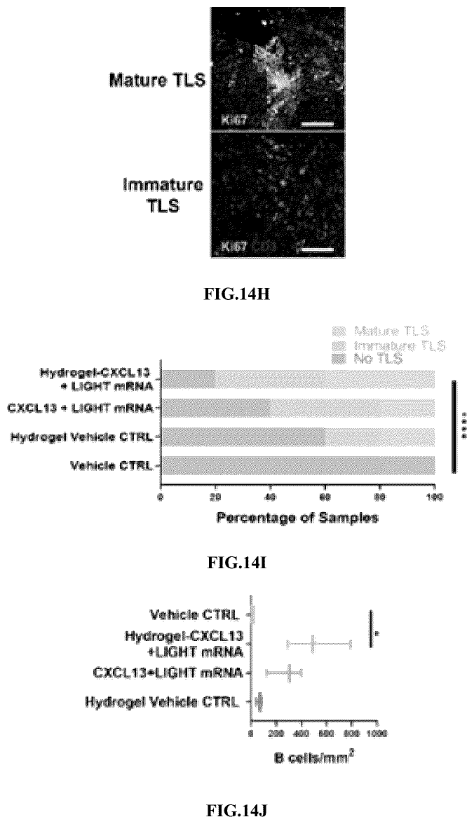

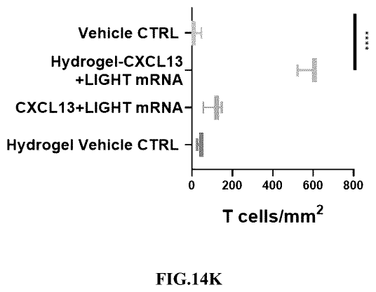

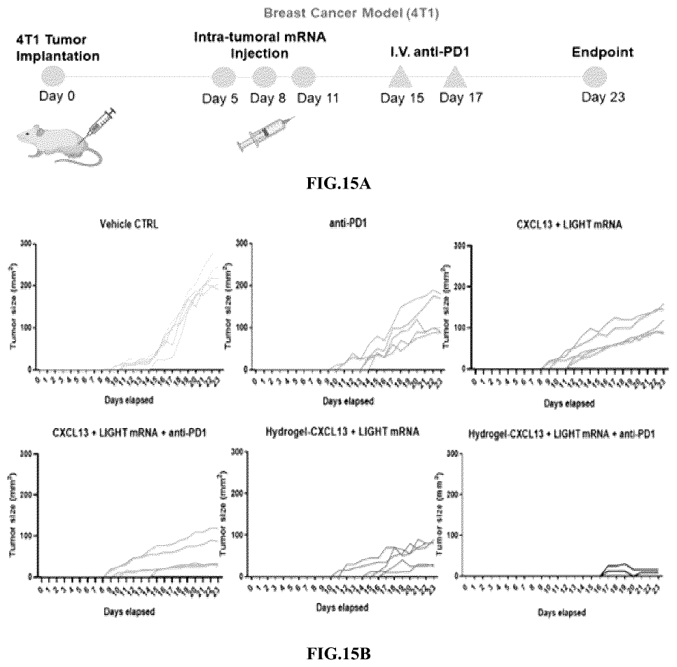

For the colon cancer model, we inoculated female BALB/c mice subcutaneously at the right flank with CT26 cells (2×10⁵ cells/animal) on day 0, followed by injections of the co-delivery hydrogel mRNA formulation (CXCL13 mRNA+LIGHT mRNA in cationic liposome), mRNA drugs alone (CXCL13 mRNA+LIGHT mRNA in cationic liposome), hydrogel control, or vehicle liposome control on days 5, 8, and 11 at the same tumor site (FIG. 14A). We observed no signs of discomfort in mice of the different experimental groups. At the endpoint (day 23), all mice were euthanized, and their tumors were extracted for further analysis. Interestingly, both the co-delivery hydrogel mRNA formulation and co-delivery of mRNA drugs alone exhibited potent primary tumor suppression (FIGS. 14B and 14C). When comparing primary tumor sizes with the hydrogel control and PBS control (FIGS. 14B and 14C), both the co-delivery hydrogel mRNA formulation and co-delivery of the mRNA drugs alone resulted in significantly smaller CT26 tumors (average tumor sizes of 135 mm^{j} and 381 mm³, respectively, compared to the hydrogel control group average tumor size of 1120.4 mm³) (FIG. 14D). This result also demonstrated that the co-delivery hydrogel mRNA formulation could better suppress tumor growth than the co-delivery of the mRNA drugs alone. Notably, the co-delivery hydrogel mRNA formulation group also significantly prolonged mouse survival (3/5 of the mice survived by the day 40 experimental endpoint) compared to the control groups (all 5 mice died) (FIG. 14E). We conducted further studies to analyze the immunological niche within the tumor of the different experimental groups. Interestingly, we predominantly observed these mature structures in the tumors of the co-delivery hydrogel mRNA formulation group (FIG. 14F). Notably, the co-delivery hydrogel mRNA formulation group generated significantly greater number of TLSs (average TLSs number of 0.43 per mm²) than the other groups (FIG. 14G). Significantly larger proportions of TLS-positive samples from the co-delivery hydrogel mRNA formulation group exhibited mature TLSs structures when compared with the PBS-control group (FIGS. 14H and 14I), along with enhanced densities of tumor-infiltrating B cells and T cells (FIGS. 14J and 14K). Overall, immunohistochemical staining CT26 results exhibit a similar trend to the previous results in the B16.f10 model.

### 6.7.3 Breast Cancer Model

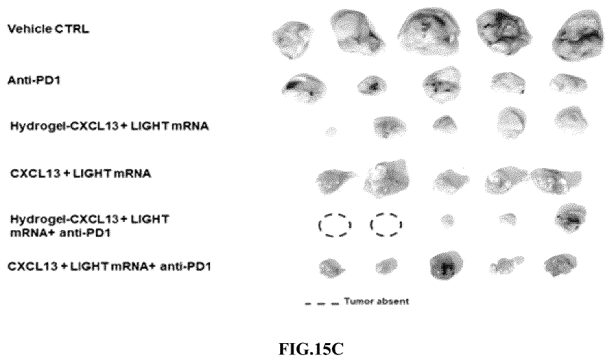

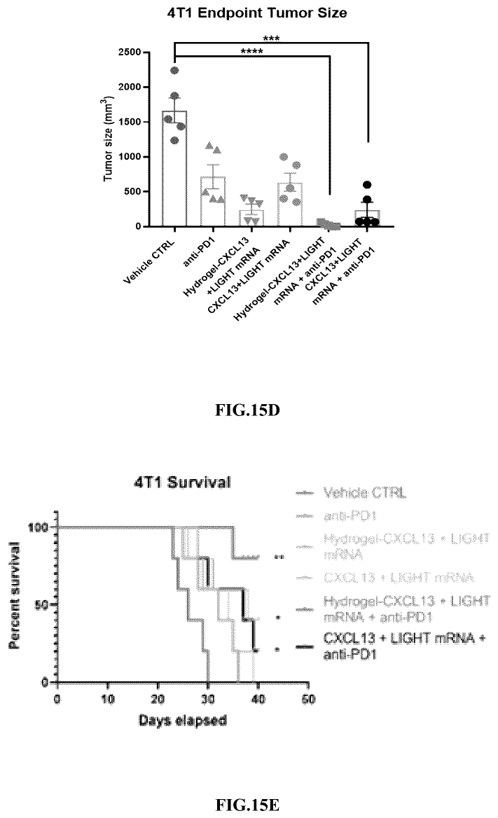

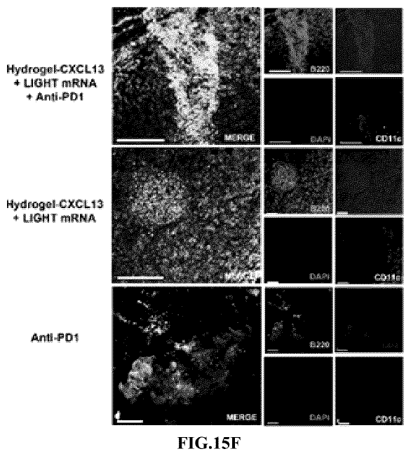

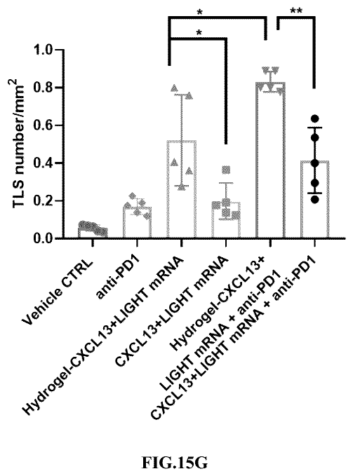

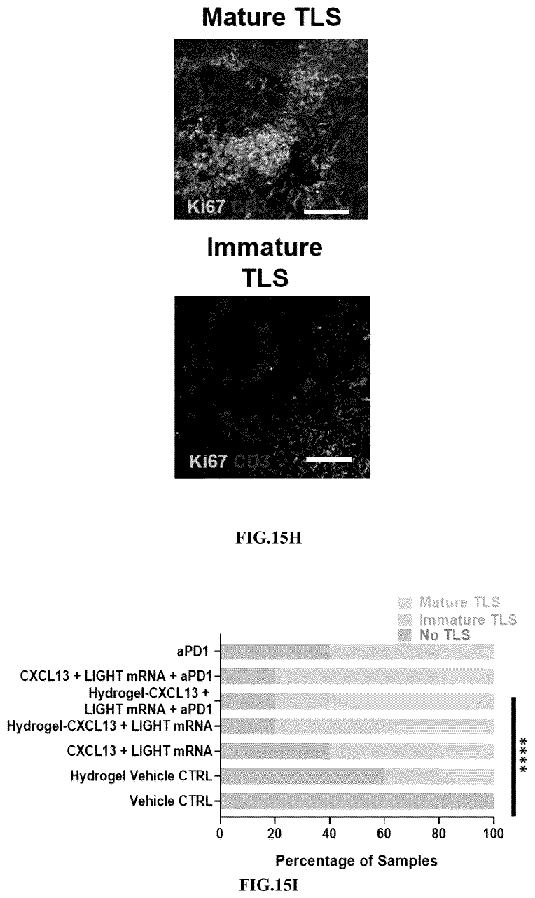

For the breast cancer model, we inoculated female BALB/c mice subcutaneously at the right flank with 4T1 cells (1×10⁵ cells/animal) on day 0, followed by injections of the co-delivery hydrogel mRNA formulation (CXCL13 mRNA+LIGHT mRNA in cationic liposome), mRNA drugs alone (CXCL13 m RNA+LIGHT mRNA in cationic liposome), or vehicle liposome control on days 5, 8, and 11 at the same tumor site. At days 15 and 17, intravenous injection of anti-PD1 therapy was administered alone or as a combination treatment (FIG. 15A). We observed no signs of discomfort in mice of the different experimental groups. At the endpoint (day 23), all mice were euthanized, and their tumors were extracted for further analysis. Interestingly, both the co-delivery hydrogel mRNA formulation plus anti-PD1 and co-delivery hydrogel mRNA formulation alone exhibited potent primary tumor suppression (FIGS. 1513 and 15C). When comparing primary tumor sizes with the vehicle control (FIGS. 15B and 15C), both the co-delivery hydrogel mRNA formulation plus anti-PD1 and co-delivery hydrogel mRNA formulation alone resulted in significantly smaller 4T1 tumors (average tumor sizes of 21.6 mm³ and 245.2 mm³, respectively, compared to the hydrogel control group average tumor size of 1665 mm³) (FIG. 15D). Both the hydrogel-based combination drugs group and the hydrogel-based mRNA formulation group outcompeted their non-hydrogel counterparts. Notably, the co-delivery hydrogel mRNA formulation plus anti-PD1 group most significantly prolonged mouse survival (1/5 of the mice survived by the day 40 experimental endpoint) compared to the control groups (all 5 mice died) (FIG. 15E). We conducted further studies to analyze the immunological niche within the tumor of the different experimental groups. Interestingly, we predominantly observed these mature structures in the tumors of the co-delivery hydrogel mRNA formulation plus anti-PD1 group and the co-delivery hydrogel mRNA formulation group (FIG. 15F). Notably, the co-delivery hydrogel mRNA formulation plus anti-PD1 group generated the greatest number of TLSs (average TLSs number of 0.83 per mm²) (FIG. 15G). Significantly larger proportions of TLS-positive samples from the co-delivery hydrogel mRNA formulation plus anti-PD1 group exhibited mature TLSs structures when compared with the other groups (FIGS. 15H and 15I). Conclusively, the results support the development of a novel injectable hydrogel-based formulation for enhancing the drug activity of mRNA therapeutics against cancer. Unique to our research, we discovered co-delivered mRNA cytokine and chemokine druggable targets capable of stimulating mature TLS formation in cancer which effectively eradicates various cancers and could enhance anti-PD1 treatment responses.

### 6.7 Discussion and Conclusion

In this study, we developed an injectable, hydrogel-based formulation for the delivery of CXCL13 chemokine and LIGHT cytokines. HA-CPP⊂CB[8]hydrogels were chosen as the drug-delivery vehicle due to their advantageous properties, including controlled drug release, biocompatibility, and porosity for cell infiltration³⁷. Specifically, HA-CPP⊂CB[8]hydrogel was utilized for encapsulating biomolecules (CXCL13, LIGHT) and serves as a suitable delivery vehicle for intraperitoneal drug administration. This drug delivery approach may overcome the limitations of reduced drug immunogenicity and rapid drug degradation, which are particularly relevant in the context of cancer, where immunosuppressive environments can hinder drug efficacy³⁸. CXCL13 and LIGHT are essential proteins involved in the generation of TLSs in non-lymphoid tissues. TLSs induction within the tumor microenvironments promotes anti-cancer immunity by maintaining and recruiting the immunostimulatory lymphocytes and upregulating central-memory T and B cells to counteract cancer progression³⁹. Therefore, the combination of a novel hydrogel drug delivery platform with critical TLS-inducing factors could enhance tumor control by creating a synthetic, anti-cancer immunological niche within the tumor microenvironment.

Using B16 melanoma mouse models, we demonstrated that a single injection of the hydrogel-based drug (CXCL13, LIGHT) formulation led to significant primary tumor site and total tumor suppression compared to PBS and vehicle controls (FIGS. 5A-5E). Although the drugs-alone group also exhibited significant tumor suppressive effects at the primary tumor site (FIG. 5C), it did not elicit significant total tumor suppression (FIG. 5D). Notably, the drug-encapsulated hydrogel formulation group prolonged mouse survival compared to control groups (FIG. 5E). To identify the immunological characteristics responsible for the treatment efficacy of the hydrogel-based drug formulation, we further analyzed the TLS organizations in B16 tumor samples. Enhanced TLS presence in tumors could serve as a predictive biomarker for a strong clinical response to cancer immunotherapies40. Previous studies have also confirmed the presence of TLS-like structures within tumor tissues derived from intraperitoneal cancer-bearing mouse models⁴¹⁻⁴². In this study, we uniquely observed enhanced TLS quantities and maturity within the tumor microenvironments after the hydrogel-based drug (CXCL13, LIGHT) treatment (FIGS. 6B and 6C), given that CXCL13 and LIGHT are known to recruit B cells and T cells towards TLS generation, respectively⁴³.

Moreover, we found that the hydrogel-based drug formulations (CXCL13, LIGHT) treatment enhances antigen-spreading (FIG. 6D and FIG. 6E). Interferon-gamma ELISPOT assay revealed that the led to enhanced OVA antigen recognition and subsequent interferon-gamma release by T cells in the intraperitoneal spleen. Elevated tumor antigen-spreading could result in increased cancer recognition by antigen-presenting cells and adaptive immune cells, which could strengthen therapeutic responses⁴⁴. Importantly, further studies showed that combining anti-PD1 drugs with a single injection of hydrogel-based drug formulation treatment could improve melanoma treatment responses and survival rates (FIGS. 7A-7E). The combination group tumors displayed enhanced TLSs presence and maturity compared to vehicle control group tumors, which positively correlates with treatment efficacy (FIGS. 8A-8C). Interestingly, anti-PD1 ICB was previously reported to increase TLS numbers via circulating T follicular helper cells in non-small cell lung cancer (NSCLC)-bearing mice⁴⁵. This may explain why a higher proportion of anti-PD1-treated mice exhibited mature and immature TLSs compared to vehicle control mice. Additionally, we observed higher percentages of CD8+ T cells in the tumor and higher percentages of CD8+ and CD4+ T cells in the spleen for the combination group compared to vehicle control group (FIGS. 8D-8F). This result suggests that T cells were key mediators of tumor eradication in the combination group, which supports the well-established role of T cell-mediated anti-cancer immunity⁴⁶. A previous study by Brenna et al. also supported the similar role of TLS generation and enhanced T cell infiltrations in facilitating cancer elimination in the context of ovarian cancers⁴⁷.

This study presents several limitations that we aim to address in future research. Firstly, the hydrogel-based drug delivery platform could be validated in combination with other ICB therapies (such as anti-CTLA-4 or anti-PDL1) across a range of cancer types⁴⁸. Secondly, gaining a deeper understanding of the underlying mechanisms would involve categorizing memory immune cell subtypes and examining the correlation between the duration of TLS persistence and favorable prognosis in various tumor mouse models and humanized mouse models⁴⁹. Thirdly, the current study is constrained by its ability to characterize the role of germinal center-like TLSs, indicative of mature and functional TLSs, in different cancer types and disease progression stages. Acquiring such insights is crucial for determining whether TLS-inducing drugs could be utilized universally at any disease stage⁵⁰. To address this issue, high-sensitivity characterization of mature TLSs could be investigated at various time points using imaging mass cytometry and multiplex immunohistochemistry. However, more cost-effective and time-effective analytic methods have not yet been developed⁵¹.

In conclusion, we have reported the development of a novel, hydrogel-based drug (CXCL13, LIGHT) formulation that effectively suppresses B16 melanoma tumor growth and improves the survival of tumor-bearing mice. Moreover, the treatment stimulated enhanced mature TLS formation, providing valuable insights regarding the generation of mature TLS through combining the LIGHT cytokine and the CXCL13 chemokine. The combination of the hydrogel-based drug with ICB also augmented therapeutic responses. We anticipate the hydrogel-based drug (CXCL13, LIGHT) formulation to be potentially useful as a therapeutic agent against clinical melanoma in the future.

### 6.8 Materials and Methods

### HA-CPP Synthesis

In a 20 ml sealed tube, 4-(4-chlorophenyl)pyridine (CPP) (378 mg, 2 mmol) and 3-chloropropylamine (260 mg, 2 mmol) were added together with 5 ml of isopropyl alcohol as the solvent. The solution was purged with nitrogen gas for 15 minutes, and the tube was tightly sealed. The reaction mixture was heated to 90° C. in an oil bath and allowed to react for 12 hours. After completion, the reaction mixture was cooled to room temperature, and the precipitated solid was filtered. The obtained solid was further recrystallized using a mixture of dichloromethane (DCM) and hexane, resulting in the formation of the final product, CPP—NH₂. Next, CPP—NH₂ (53 mg, 0.15 eq) and hyaluronic acid (HA, molecular weight of 60 k) (0.5 g, 1 eq) were dissolved in 50 ml of MES buffer (pH 5). 4-(4,6-dimethoxy-1,3,5-triazin-2-yl)-4-methyl morpholinium chloride (DMTMM) (0.343 g, 1 eq) was then added to the solution. The mixture was vigorously stirred for 48 hours. After completion of the reaction, dialysis was performed using salt water and then DI water for a total duration of 3 days. Finally, the remaining solution was lyophilized to obtain the solid final product HA-CPP.

### Hydrogel Formation

Lyophilized HA-CPP was dissolved in DI water and the pH of the dissolved solutions was balanced to neutrality by adding either 0.1 M NaOH or HCl in small amounts as necessary. These solutions were then combined with CB[8] at a stoichiometric ratio, leading to the formation of hydrogel through non-covalent bonding.

### Dynamic Oscillatory Rheology

The rheological properties of 2 wt % HA-CPP⊂CB[8]hydrogels were studied using a Modular Compact Rheometer (Anton Parr), with a 25 mm parallel plate and a gap set to 0.5 μm. An initial amplitude sweep with parameters 10 rad/s and 0.01-200% strain was executed to ensure that the later frequency sweep (using 1% strain and a range of 0.1-200 rad/s) happened within the linear viscoelastic region. The shear viscosity measurements were performed on hydrogels with a shear rate ramping from 0.1 to 100 s−1. Additionally, step-strain tests were performed using a cyclic strain fluctuating between 1 and 100% at 10 rad/s.

### Animal Experiments

Four weeks-old female C57BL/6J mice were provided and housed by the Centre of Comparative Medicine at Laboratory block, the University of Hong Kong. The murine-derived B16-OVA cell-line was cultured for three passages at a maximum of 85% confluency in 10% fetal bovine serum (FBS) in Dulbecco's modified Eagle medium (DMEM) with 100 μg/mL penicillin/streptomycin and tested negative for mycoplasma. Cells were suspended in filtered PBS. A total of 3×10⁵ cells in 100 μL were injected into the intraperitoneal cavity of female C57BL/6 mice using a Hamilton syringe and size 25S gauge bevel tip needle. A 2 wt % HA-CPP⊂CB[8]hydrogel, hydrogel-encapsulating protein drugs (CXCL13, ACROBiosystems and recombinant LIGHT, Rndsystems), and anti-PD1 (Invivomab) treatment diluted in PBS were injected into the intraperitoneal cavity of female C57BL/6 mice using a Hamilton syringe and size 22S gauge bevel tip needle. Animals were euthanized according to strict humane endpoint guidelines and judged based on the veterinarian's observation for signs and symptoms of pain and distress to minimize harm to tumor-bearing animals. All animal experiments in this study were approved by the Committee on the Use of Live Animals in Teaching & Research, the University of Hong Kong (#CULATR 23-034).

At the tumor endpoint, mice were sacrificed, and primary and metastatic tumors were surgically harvested for tumor size analysis using electronic calipers and tumor weight analysis using a mass balance. Spleens were also surgically harvested and processed for further analysis by resuspending strained splenocytes in an FBS-containing RPMI medium, adding red blood cell lysis buffer, and washing in filtered PBS. Harvested tumors were processed overnight in 4% paraformaldehyde followed by resuspension in 30% sucrose for immunohistochemistry. Harvested tumors were also processed into single-cell suspension by a Tumor dissociation kit (Miltenyi Biotec, 130-096-730) according to the manufacturer's protocol for flow cytometry. For survival studies, tumor-bearing mice were closely monitored up to the day 40 endpoint and euthanized based on the veterinarian's suggestions, considering the observation of signs and symptoms of pain and distress to minimize harm.

### Immunohistochemical Staining

Five μm cryosection slices were prepared from OCT-preserved tissues and mounted on glass slides. Immunohistochemistry was performed following the manufacturer's instructions using multiple-antibody staining protocols. Briefly, the slides were washed in filtered PBS and blocked with PBS containing 5% FBS for 1 hour. The following primary monoclonal antibodies were used: rabbit anti-mouse CD3 (Invitrogen, 1:25), rat anti-mouse B220 (Invitrogen, 1:25), and hamster anti-mouse APC-CD11c (BioLegend, 1:25). Primary antibodies were incubated in the dark at 4° C. overnight and washed several times using PBS-Tween. The following secondary monoclonal antibodies were used: donkey anti-rat 647-antibody (Invitrogen, 1:50), goat anti-rabbit FITC-antibody (Invitrogen, 1:50). Secondary antibodies were incubated in the dark for 1-hour incubation at room temperature and washed several times using PBS-Tween. DAPI-infused mounting media (Abcam, ab104139) was used to mount the slides following the manufacturer's protocol. Images were taken on the confocal microscope LSM900 and processed using ImageJ (v1.53k). For TLS quantifications, sections were scanned and analyzed using Zen, ImageJ software, or QuPath.

### Criteria for TLSs Definition

Murine TLSs were identified by immunostaining of sections for co-presence of CD3+, B220+, and CD11+ cells in a compact organization. Mature TLSs were characterized by a clear segregation of the CD3+ zone with adjacent CD11c cells surrounding the B220+ zones in a germinal center-like compact organization. Immature TLSs were described as the co-presence of CD3+ zones adjacent to the compact cluster of B220+ zones. The absence of TLSs was defined as the lack of CD3+, B220+, or CD11+ cells clustering in a compact organization.

### Flow Cytometric Analysis

Murine tumors and spleens were processed into single-cell suspensions after passing through a 70-μm cell strainer (Thermo Fisher Scientific). At 4° C., 0.5-2×10⁶ cells were incubated with Block buffer (1×PBS, 2% FBS, 0.5% BSA) for 15 minutes, followed by the addition of 50 μl Master Mix surface marker staining (FITC anti-mouse CD3, PE/Cy7 anti-mouse CD4, BV-421 anti-mouse CD8a, 1:50, BioLegend) for 30 minutes. Cells were then washed in filtered PBS and centrifuged at 2000 RPM prior to resuspension in 200 μl FACs buffer (1×PBS, 2% FBS, 0.002% Sodium Azide). For fluorescence-activated cell sorting, a NovoCyte Quanteon Flow cytometer was used to analyze the samples.

### IFN-γ ELISPOT Assay

Mice were sacrificed, and spleen cells were collected for IFN-7 ELISPOT analysis according to the manufacturer's protocol (Sigma-Aldrich). Briefly, 100 μl of spleen cells were incubated on the IFN-γELISPOT plate, which was pre-activated for 30 minutes using 200 μl DMEM media without FBS. Subsequently, 5 μg of the OVA peptide (OVA 257-264 and the positive inducer) were added, and cells were incubated for 20 hours at 37° C. in 5% CO2. After disturbing the media, cells were washed in a washing buffer and incubated with the secondary biotinylated antibody for 1 hour at RT. Finally, cells were incubated with the biotin substrate, followed by washing and drying to yield visible spots in positive wells.

### Statistical Analysis

All results were plotted in Prism 7 (GraphPad Software Inc). Statistical comparisons between groups were determined by unpaired t-test using Prism 7. Chi-squared contingency tests were used for TLS grading. Mantel-Cox tests were used for survival analysis. For all tests, p<0.05 was considered statistically significant.

Exemplary products, systems and methods are set out in the following items:
1. A compound comprising hyaluronic acid and 4-(4-chlorophenyl)pyridine (HA-CPP) or a salt thereof.
2. The compound of item 1, wherein the HA and CPP are linked directly.
3. The compound of item 1 or 2, wherein the HA and CPP are linked through a linker.
4. The compound of item 3, wherein the linker is an alkyleneamine.
5. The compound of item 3 or 4, wherein the linker is —(CH₂)₃—NH—.
6. The compound of any one of the preceding items, wherein about 10-30% of hyaluronic acid disaccharide subunits are substituted with CPP.
7. The compound of item 6, wherein about 15% of hyaluronic acid disaccharide subunits are substituted with CPP.
8. The compound of any one of the preceding items, comprising at least one moiety of formula (1):

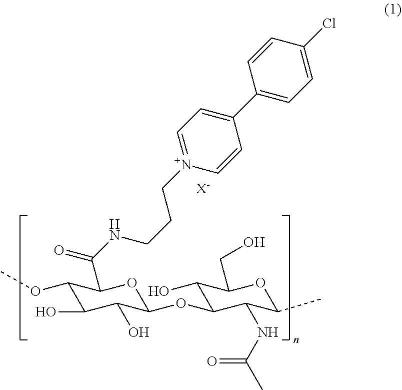

wherein n is an integer and X is a counter-ion;
or a salt thereof.
9. The compound of item 8, comprising about 10-30% of a moiety of formula (1) wherein n is 1.
10. The compound of item 8 or 9, comprising about 15% of the moiety of formula (1) wherein n is 1.
11. A composition comprising the compound of any one of the preceding items-10, and a cucurbituril.
12. The composition of item 11, wherein the cucurbituril is a cucurbit[n]uril (CB[n]), wherein n is 5, 6, 7 or 8.
13. The composition of items 11-12, wherein n is 8 (HA-CPP⊂CB[8]).
14. The composition of any one of items 11-13, in the form of a hydrogel.
15. The composition of any one of items 11-14, wherein the composition is an injectable composition.
16. The composition of any one of items 11-15, comprising water at a weight ratio of about 1-5%.
17. The composition of any one of items 11-16, wherein the HA-CPP and CB[n] are linked through non-covalent binding.
18. The composition of any one of items 11-17, further comprising one or more biologically active agents.
19. The composition of any one of items 11-18, wherein HA-CPP⊂CB[n] is combined with the biologically active agent through non-covalent binding to form a complex.
20. The composition of any one of items 11-19, comprising HA-CPP⊂CB[8] and one or more biologically active agent.
21. The composition of any one of items 11-20, wherein HA-CPP⊂CB[8] is combined with the biologically active agent through non-covalent binding to form a complex.
22. The composition of any one of items 11-21, wherein the biologically active agent is a chemokine, cytokine, immune-checkpoint inhibitor, cancer vaccine, chimeric antigen receptor or a combination thereof.
23. The composition of any one of items 11-22, wherein the biologically active agent is CXCL13, CCL21, IL-4, IL-7, IL-2, LIGHT, CCL19, CXCL12, CXCL13, lymphotoxin, TNF-α or a combination thereof.
24. The composition of any one of items 11-23, wherein the chemokine is CXCL13 and the cytokine is LIGHT.
25. A method of treating cancer comprising administering the composition of any one of items 11-24 to a subject in need thereof.
26. The method of item 25, wherein the composition is administered by injection to form tertiary lymphoid structures and augmentation of immune checkpoint blockade.
27. The method of item 25 or 26, wherein the composition suppresses tumor growth, prolongs survival, increases TLS density, promotes TLS maturation, up-regulates OVA antigen-spreading in secondary lymphoid organs or a combination thereof.
28. The method of item 25, 26 or 27, further comprising administering an anti-PD1 drug, anti-CTLA-4 drug, anti-PD1, anti-PDL1, anti-CTLA-4 or a combination thereof.
29. The method of item 25, 26 or 27, wherein the administering comprises direct intratumoral injection.
30. The method of item 25, 26 or 27, wherein the cancer is melanoma, colorectal cancer, lung cancer, pancreatic cancer, oral squamous cell carcinoma, or invasive breast cancer.
31. The method of item 25, 26 or 27, wherein the subject is human, feline, canine, porcine, rabbit or rodent.
32. A method of making HA-CPP comprising: (a) reacting 4-(4-chlorophenyl)pyridine with a compound of the formula X—(CH₂)₃—NH₂ wherein X is a leaving group; and (b) reacting the product of step (a) with hyaluronic acid (HA) having molecular weight of about 6 kDa to 1000 kDa.
33. A method of making HA-CPP comprising: (i) reacting 4-(4-chlorophenyl)pyridine with 3-halopropylamine to form a pyridinium salt CPP—NH₂ and (ii) performing an amidation reaction with at least one glucuronic acid group of hyaluronic acid (HA), wherein the HA has a molecular weight of about 6 kDa to 1000 kDa.
34. The method of item 33, wherein the halo is chloro.
35. The method of item 32, 33 or 34, further comprising combining HA-CPP and cucurbit[n]uril (CB[n]) wherein n is 5, 6, 7 or 8 to form HA-CPP⊂CB[n].
36. The method of item 32, 33, 34 or 35, further comprising combining HA-CPP and cucurbit[8]uril (CB[8]) to form HA-CPP⊂CB[8].
37. The method of any one of items 32-36, wherein the stoichiometric ratio between CPP and CB[8] is 2:1 or greater than 2:1.
38. The method of any one of items 32-37, wherein the degree of substitution of CPP is about 10 to about 30%.
39. The method of any one of items 32-38, wherein the degree of substitution of CPP is about 15%.

The foregoing description of the specific embodiments will so fully reveal the general nature of the disclosure that others can, by applying knowledge within the skill of the relevant art(s) (including the contents of the documents cited and incorporated by reference herein), readily modify and/or adapt for various applications such specific embodiments, without undue experimentation, without departing from the general concept of the present disclosure. Such adaptations and modifications are therefore intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one skilled in the relevant art(s).

While various embodiments of the present disclosure have been described above, it should be understood that they have been presented by way of examples, and not limitation. It would be apparent to one skilled in the relevant art(s) that various changes in form and detail could be made therein without departing from the spirit and scope of the disclosure. Thus, the present disclosure should not be limited by any of the above-described exemplary embodiments but should be defined only in accordance with the following claims and their equivalents.

All references cited herein are incorporated herein by reference in their entirety and for all purposes to the same extent as if each individual publication or patent or patent application was specifically and individually indicated to be incorporated by reference in its entirety for all purposes.

### REFERENCES

1. Gray-Schopfer, V., Wellbrock, C., Marais, R. Melanoma biology and new targeted therapy. Nature. 445, 851-857 (2007).
2. Wan, G., Nguyen, N., Liu, F. et al. Prediction of early-stage melanoma recurrence using clinical and histopathologic features. npj Precis. Onc. 6, 79 (2022).
3. Ahmed, B., Qadir, M. I., Ghafoor, S. Malignant Melanoma: Skin Cancer-Diagnosis, Prevention, and Treatment. Crit Rev Eukaryot Gene Expr. 30(4), 291-297 (2020).
4. Pavri, S. N., Clune, J., Ariyan, S., Narayan, D. Malignant Melanoma: Beyond the Basics. Plast Reconstr Surg. 138(2), 330e-340e (2016).
5. Kim, S. K., Cho, S. W. The Evasion Mechanisms of Cancer Immunity and Drug Intervention in the Tumor Microenvironment. Front Pharmacol. 13, 868695 (2022).
6. Vinay, D. S., Ryan, E. P., Pawelec, G. et al. Immune evasion in cancer: Mechanistic basis and therapeutic strategies. Semin Cancer Biol. 35, Suppl:S185-S198 (2015).
7. Sato, Y., Silina, K., van den Broek, M. et al. The roles of tertiary lymphoid structures in chronic diseases. Nat Rev Nephrol (2023).
8. Helmink, B. A. et al. B cells and tertiary lymphoid structures promote immunotherapy response. Nature. 577(7791), 549-555 (2020).
9. Bery, A. I., Shepherd, H. M., Li, W., Krupnick, A. S., Gelman, A. E., Kreisel, D. Role of tertiary lymphoid organs in the regulation of immune responses in the periphery. Cell Mol Life Sci. 79, 359 (2022).
10. Munoz-Erazo, L., Rhodes, J. L., Marion, V. C., Kemp, R. A. Tertiary lymphoid structures in cancer—considerations for patient prognosis. Cell Mol Immunol. 17(6), 570-575 (2020).
11. Meylan, M. ert al. Tertiary lymphoid structures generate and propagate anti-tumor antibody-producing plasma cells in renal cell cancer. Immunity. 55(3):527-541.e5 (2022).
12. Fridman, W. H., Meylan, M., Petitprez, F., Sun, C. M., Italiano, A., Sautes-Fridman, C. B cells and tertiary lymphoid structures as determinants of tumour immune contexture and clinical outcome. Nat Rev Clin Oncol. 19(7):441-457 (2022).
13. Paijens, S. T., Vledder, A., de Bruyn, M., Nijman, H. W. Tumor-infiltrating lymphocytes in the immunotherapy era. Cell Mol Immunol. 18(4):842-859 (2021).
14. Schumacher, T. N., Thommen, D. S. Tertiary lymphoid structures in cancer. Science. 375(6576), eabf9419 (2022).
15. Cabrita, R. et al. Tertiary lymphoid structures improve immunotherapy and survival in melanoma. Nature. 577(7791), 561-565 (2020).
16. Pimenta, E. M., Barnes, B. J. Role of Tertiary Lymphoid Structures (TLS) in Anti-Tumor Immunity: Potential Tumor-Induced Cytokines/Chemokines that Regulate TLS Formation in Epithelial-Derived Cancers. Cancers (Basel). 6(2), 969-97 (2014).
17. Qin, M., Jin, Y., Pan, L. Y. Tertiary lymphoid structure and B-cell-related pathways: A potential target in tumor immunotherapy. Oncol Lett. 22(6), 836 (2021).
18. Groeneveld, C. S., Fontugne, J., Cabel, L., Bernard-Pierrot, I., Radvanyi, F., Allory, Y., de Reynies, A. Tertiary lymphoid structures marker CXCL13 is associated with better survival for patients with advanced-stage bladder cancer treated with immunotherapy. Eur J Cancer. 148, 181-189 (2021).
19. Johansson-Percival, A., Ganss, R. Therapeutic Induction of Tertiary Lymphoid Structures in Cancer Through Stromal Remodeling. Front Immunol. 12, 674375 (2021).
20. Johansson-Percival, A., He, B., Li, Z J. et al. De novo induction of intratumoral lymphoid structures and vessel normalization enhances immunotherapy in resistant tumors. Nat Immunol 18, 1207-1217 (2017).
21. Chaurio, R. A. et al. TGF-β-mediated silencing of genomic organizer SATB1 promotes Tfh cell differentiation and formation of intra-tumoral tertiary lymphoid structures. Immunity. 55(1), 115-128.e9 (2022).
22. Chelvanambi, M., Fecek, R. J., Taylor, J. L., Storkus, W. J. STING agonist-based treatment promotes vascular normalization and tertiary lymphoid structure formation in the therapeutic melanoma microenvironment. J Immunother Cancer. 9(2), e001906 (2021).
23. Overacre-Delgoffe, A. E., Bumgarner, H. J., Cillo, A. R., Burr, A. H. P., Tometich, J. T., Bhattacharjee, A., Bruno, T. C., Vignali, D. A. A., Hand, T. W. Microbiota-specific T follicular helper cells drive tertiary lymphoid structures and anti-tumor immunity against colorectal cancer.

Immunity. 54(12), 2812-2824.e4 (2021).
24. Horeweg, N. et al. Tertiary lymphoid structures critical for prognosis in endometrial cancer patients. Nat Commun. 13(1), 1373 (2022).
25. Najibi, A. J., Mooney, D. J. Cell and tissue engineering in lymph nodes for cancer immunotherapy. Adv Drug Deliv Rev. 161-162:42-62 (2020).
26. Abbott, M., Ustoyev, Y. Cancer and the Immune System: The History and Background of Immunotherapy. Semin Oncol Nurs. 35(5), 150923 (2019).
27. Li, H., Ding, J. Y., Zhang, M. J., Yu, H. J., Sun, Z. J. Tertiary lymphoid structures and cytokines interconnections: The implication in cancer immunotherapy. Cancer Lett. 568:216293 (2023).
28. Bhat, A. A. et al. Cytokine- and chemokine-induced inflammatory colorectal tumor microenvironment: Emerging avenue for targeted therapy. Cancer Commun (Lond). 42(8):689-715 (2022).
29. Lei, L., Huang, D., Gao, H., He, B., Cao, J. Peppas NA. Hydrogel-guided strategies to stimulate an effective immune response for vaccine-based cancer immunotherapy. Sci Adv. 8(47):eadc8738 (2022).
30. Hoffman, A. S. Hydrogels for biomedical applications. Adv Drug Deliv Rev. 54(1):3-12 (2002).
31. Bertsch, P., Diba, M., Mooney, D. J., Leeuwenburgh, S. C. G. Self-Healing Injectable Hydrogels for Tissue Regeneration. Chem Rev. 123(2):834-873 (2023).
32. Li, J., Mooney, D. J. Designing hydrogels for controlled drug delivery. Nat Rev Mater. 1(12) (2016).
33. Wang, H., Mooney, D. J. Biomaterial-assisted targeted modulation of immune cells in cancer treatment. Nat Mater. 17(9), 761-772 (2018).
34. Melero, I., Gaudernack, G., Gerritsen, W. et al. Therapeutic vaccines for cancer: an overview of clinical trials. Nat Rev Clin Oncol. 11(9):509-524 (2014).
35. McFetridge, M. L., Del Borgo, M. P., Aguilar, M. I., Ricardo, S. D. The use of hydrogels for cell-based treatment of chronic kidney disease. Clin Sci (Lond). 132(17), 1977-1994 (2018).
36. Aqbi, H. F., Wallace, M., Sappal, S., Payne, K. K., Manjili, M. H. IFN-7 orchestrates tumor elimination, tumor dormancy, tumor escape, and progression. J Leukoc Biol. 10.1002/JLB.5MIR0917-351R (2018).
37. Narayanaswamy, R., Torchilin, V. P. Hydrogels and Their Applications in Targeted Drug Delivery. Molecules. 24(3), 603 (2019).
38. Lei, L., Huang, D., Gao, H., He, B., Cao, J., Peppas, N. A. Hydrogel-guided strategies to stimulate an effective immune response for vaccine-based cancer immunotherapy. Sci Adv. 8(47), eadc8738 (2022).
39. Dieu-Nosjean, M. C., Giraldo, N. A., Kaplon, H., Germain, C., Fridman, W. H., Sautes-Fridman, C. Tertiary lymphoid structures, drivers of the anti-tumor responses in human cancers. Immunol Rev. 271(1), 260-75 (2016).
40. Triib, M., Zippelius, A. Tertiary Lymphoid Structures as a Predictive Biomarker of Response to Cancer Immunotherapies. Front Immunol. 12, 674565 (2021).
41. Klein, C., Devi-Marulkar, P., Dieu-Nosjean, M. C., Germain, C. Development of Tools for the Selective Visualization and Quantification of TLS-Immune Cells on Tissue Sections. Methods Mol Biol. 1845, 47-69 (2018).
42. Rodriguez, A. B. et al. Immune mechanisms orchestrate tertiary lymphoid structures in tumors via cancer-associated fibroblasts. Cell Rep. 36(3), 109422 (2021).
43. Johansson-Percival, A., Ganss, R. Therapeutic Induction of Tertiary Lymphoid Structures in Cancer Through Stromal Remodeling. Front Immunol. 12, 674375 (2021).
44. Pan, J., Zhang, Q., Sei, S., Shoemaker, R. H., Lubet, R. A., Wang, Y., You, M. Immunoprevention of KRAS-driven lung adenocarcinoma by a multipeptide vaccine. Oncotarget. 8, 82689-82699 (2017).
45. Sinchez-Alonso, S., Setti-Jerez, G., Arroyo, M. et al. A new role for circulating T follicular helper cells in humoral response to anti-PD-1 therapy. Journal for ImmunoTherapy of Cancer. 8, e001187 (2020).
46. Waldman, A. D., Fritz, J. M., Lenardo, M. J. A guide to cancer immunotherapy: from T cell basic science to clinical practice. Nat Rev Immunol. 20, 651-668 (2020).
47. Brenna, E., Pedroza-Pacheco, I. Harnessing CXCL13 in ovarian cancer. Nat Rev Immunol. 22, 145 (2022).
48. Cai, D., Yu, H., Wang, X., Mao, Y., Liang, M., Lu, X., Shen, X., Guan, W. Turning Tertiary Lymphoid Structures (TLS) into Hot Spots: Values of TLS in Gastrointestinal Tumors. Cancers. 15(2), 367 (2023).
49. He, M., He, Q., Cai, X. et al. Intratumoral tertiary lymphoid structure (TLS) maturation is influenced by draining lymph nodes of lung cancer Journal for ImmunoTherapy of Cancer. 11, e005539 (2023).
50. Andrew, J. G. et al. Germinal center reactions in tertiary lymphoid structures associate with neoantigen burden, humoral immunity and long-term survivorship in pancreatic cancer. Oncolmmunol. 10,1 (2021).
51. Le Rochais, M., Hémon, P., Ben-Guigui, D., Garaud, S., Le Dantec, C., Pers, J. O., Cornec, D., Uguen, A. Deciphering the maturation of tertiary lymphoid structures in cancer and inflammatory diseases of the digestive tract using imaging mass cytometry. Front Immunol. 14, 1147480 (2023).
52. R. Narayanaswamy, V. P. Torchilin, Hydrogels and Their Applications in Targeted Drug Delivery, Molecules 24 (2019).
53. M. Meylan, F. Petitprez, E. Becht, A. Bougoiiin, G. Pupier, A. Calvez, I. Giglioli, V. Verkarre, G. Lacroix, J. Verneau, C. M. Sun, P. Laurent-Puig, Y. A. Vano, R. Elaidi, A. Méjean, R. Sanchez-Salas, E. Barret, X. Cathelineau, S. Oudard, C. A. Reynaud, A. de Reyniès, C. Sautès-Fridman, W. H. Fridman, Tertiary lymphoid structures generate and propagate anti-tumor antibody-producing plasma cells in renal cell cancer, Immunity 55 (2022) 527-541.e5.
54. H. Li, J. Y. Ding, M. J. Zhang, H. J. Yu, Z. J. Sun, Tertiary lymphoid structures and cytokines interconnections: The implication in cancer immunotherapy, Cancer Lett. 568 (2023) 216293.
55. C. S. Groeneveld, J. Fontugne, L. Cabel, I. Bernard-Pierrot, F. Radvanyi, Y. Allory, A. de Reyniès, Tertiary lymphoid structures marker CXCL13 is associated with better survival for patients with advanced-stage bladder cancer treated with immunotherapy, Eur. J. Cancer 148 (2021) 181-189.
56. A. Johansson-Percival, R. Ganss, Therapeutic Induction of Tertiary Lymphoid Structures in Cancer Through Stromal Remodeling, Front. Immunol. 12 (2021) 674375.

What is claimed:

1. A compound comprising hyaluronic acid and 4-(4-chlorophenyl)pyridine (HA-CPP) or a salt thereof.

2. The compound of claim 1, wherein the HA and CPP are linked through a linker, wherein the linker is an alkyleneamine.

3. The compound of claim 2, wherein the linker is —(CH₂)₃—NH—.

4. The compound of claim 1, wherein about 10-30% of hyaluronic acid disaccharide subunits are substituted with CPP.

5. The compound of claim 1, comprising at least one moiety of formula (1): 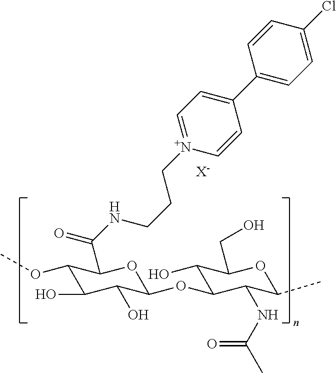
wherein n is an integer and X is a counter-ion;
or a salt thereof.

6. A composition comprising the compound of claim 1, and a cucurbit[n]uril (CB[n]), wherein in is 5, 6, 7 or 8 (HA-CPP⊂CB[n]).

7. The composition of claim 6, wherein n is 8 (HA-CPP⊂CB[8]).

8. The composition of claim 6, in the form of a hydrogel.

9. The composition of claim 6, wherein the composition is an injectable composition and comprises water at a weight ratio of about 1-5%.

10. The composition of claim 6, wherein the HA-CPP and CB[n] are linked through non-covalent binding.

11. The composition of claim 6, further comprising one or more biologically active agents.

12. The composition of claim 11, wherein HA-CPP⊂CB[n] is combined with the biologically active agent through non-covalent binding to form a complex.

13. The composition of claim 6, comprising HA-CPP⊂CB[8] and one or more biologically active agent.

14. The composition of claim 13, wherein HA-CPP⊂CB[8] is combined with the biologically active agent through non-covalent binding to form a complex.

15. The composition of claim 11, wherein the biologically active agent is a chemokine, cytokine, immune-checkpoint inhibitor, cancer vaccine, chimeric antigen receptor or a combination thereof.

16. The composition of claim 11, wherein the biologically active agent is CXCL13, CCL21, IL-4, IL-7, IL-2, LIGHT, CCL19, CXCL12, CXCL13, lymphotoxin, TNF-α or a combination thereof.

17. The composition of claim 16, wherein the chemokine is CXCL13 and the cytokine is LIGHT.

18. A method of treating cancer comprising administering the composition of claim 10 to a subject in need thereof.

19. The method of claim 18, wherein the composition is administered by injection to form tertiary lymphoid structures and augmentation of immune checkpoint blockade.

20. The method of claim 18, wherein the composition suppresses tumor growth, prolongs survival, increases TLS density, promotes TLS maturation, up-regulates OVA antigen-spreading in secondary lymphoid organs or a combination thereof.

21. The method of claim 18, further comprising administering an anti-PD1 drug, anti-CTLA-4 drug, anti-PD1, anti-PDL1, anti-CTLA-4 or a combination thereof.

22. The method of claim 18, wherein the administering comprises direct intratumoral injection.

23. The method of claim 18, wherein the cancer is melanoma, colorectal cancer, lung cancer, pancreatic cancer, oral squamous cell carcinoma, or invasive breast cancer.
